(19) United States
(12) Patent Application Publication
(10) Pub. No.: US 2024/0060121 A1
(43) Pub. Date: Feb. 22, 2024

(54) METHODS FOR MULTI-FOCAL IMAGING FOR MOLECULAR PROFILING

(71) Applicants: President and Fellows of Harvard College, Cambridge, MA (US); Children's Medical Center Corporation, Boston, MA (US)

(72) Inventors: Jeffrey R. Moffitt, (US); Hazen P. Babcock, Cambridge, MA (US)

(73) Assignees: President and Fellows of Harvard College, Cambridge, MA (US); Children's Medical Center Corporation, Boston, MA (US)

(21) Appl. No.: 17/769,536

(22) PCT Filed: Nov. 19, 2020
(86) PCT No.: PCT/US2020/061254

§ 371 Date: Apr. 15, 2022

Related U.S. Application Data
(60) Provisional application No. 62/938,194, filed on Nov. 20, 2019.

(51) Int. Cl. C12Q 1/6841; G01N 21/64

(52) U.S. Cl. CPC C12Q 1/6841; G01N 21/6458; C12Q 2600/166

(57) ABSTRACT

The present disclosure generally relates to systems and methods for multi-focal imaging, for example, for determining nucleic acids in cells or other samples. In some cases, multiple focal planes may simultaneously be determined, e.g., by using a plurality of detectors, such as a plurality of cameras, which image the same sample, but at least some of which are focused on different focal planes within the sample. Thus, the sample may be imaged in 3 dimensions, e.g., without sample refocusing. In certain cases, this may improve the resolution of imaging, in space and/or time. Various embodiments can be used to increase imaging throughput and/or resolution in image-based approaches, e.g., for single-cell molecular profiling such as multiplexed error robust fluorescence in situ hybridization (MERFISH), or for other applications.

### RELATED APPLICATIONS

This application claims the benefit of U.S. Provisional Patent Application Ser. No. 62/938,194, filed Nov. 20, 2019, entitled “Systems and Methods for Multi-Focal Imaging for Molecular Profiling,” by Moffitt, et al., incorporated herein by reference in its entirety.

### FIELD

The present disclosure generally relates to systems and methods for multi-focal imaging.

### BACKGROUND

Methods for deep molecular profiling of single cells and other biological samples via massively multiplexed in situ imaging have emerged as powerful methods for addressing a wide variety of biological questions. In these approaches, the two-dimensional distribution of a large number of distinct molecular targets are determined within biological samples via multiplexed wide-field imaging modalities and such measurements are extended to determine the three-dimensional distribution of samples by imaging of multiple distinct focal planes, i.e. the collection of z-stacks. However, the throughput—the volume of sample or number of cells that can be characterized per unit time—is often limited by the speed with which the sample can be imaged and the speed with which the sample can be refocused. Similarly, the resolution of distinct molecular species in these samples can often be limited by the substantially larger extent of the optical-point-spread-function along the axial axis. Thus, there is a need for improved imaging modalities that allow more images to be collected per unit time to increase the throughput of such samples, and/or which can provide increased optical resolution along the z-axis.

### SUMMARY

The present disclosure generally relates to systems and methods for multi-focal imaging. The subject matter of the present disclosure involves, in some cases, interrelated products, alternative solutions to a particular problem, and/or a plurality of different uses of one or more systems and/or articles.

One set of embodiments is generally directed to a method comprising exposing a sample to a plurality of nucleic acid probes; for each of the nucleic acid probes, capturing images of the sample using at least 4 detectors focused on different focal planes within the sample; determining binding of the nucleic acid probes within the sample using the images; and determining an abundance and/or a spatial distribution of nucleic acids within the sample corresponding to the binding of the plurality of nucleic acid probes.

Another set of embodiments is generally directed to a method comprising exposing a sample to a plurality of nucleic acid probes; for each of the nucleic acid probes, exposing the nucleic acid probes to amplifier nucleic acids able to bind thereto, wherein a maximum finite number of amplifier nucleic acids is able to directly or indirectly bind to a nucleic acid probe; and for each of the nucleic acid probes, capturing images of the sample using at least 4 cameras focused on different focal planes within the sample.

In yet another set of embodiments, the method comprises analyzing a sample using MERFISH, where the act of analyzing comprises capturing images using a plurality of cameras. The method, in still another aspect, comprises determining an abundance and/or a spatial distribution of nucleic acids within a sample using a plurality of cameras. According to yet another aspect, the method comprises determining, optionally simultaneously, a copy number and/or a spatial distribution of hundreds to thousands of nucleic acid species, such as RNA and/or DNA, in samples such as single cells.

In another aspect, the present disclosure encompasses methods of making one or more of the embodiments described herein, for example, a device for multi-focal imaging. In still another aspect, the present disclosure encompasses methods of using one or more of the embodiments described herein, for example, a device for multi-focal imaging.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a schematic illustration of a device in accordance with one embodiment of the disclosure;

FIG. 2 is a schematic illustration of a device in accordance with another embodiment of the disclosure; and

FIGS. 3A-3E are schematic illustrations of an embodiment that uses primary and secondary amplifier nucleic acids to amplify a signal; and.

FIG. 4 is a schematic illustration of a device in accordance with another embodiment of the disclosure.

### DETAILED DESCRIPTION

The present disclosure generally relates to systems and methods for multi-focal imaging, for example, for determining nucleic acids in cells or other samples. In some cases, multiple focal planes may simultaneously be determined, e.g., by using a plurality of detectors, such as a plurality of cameras, which image the same sample, but at least some of which are focused on different focal planes within the sample. Thus, the sample may be imaged in 3 dimensions, e.g., without sample refocusing. In certain cases, this may improve the resolution of imaging, in space and/or time. Various embodiments can be used to increase imaging throughput and/or resolution in image-based approaches, e.g., for single-cell molecular profiling such as multiplexed error robust fluorescence in situ hybridization (MERFISH), or for other applications.

For example, certain aspects are generally directed to systems that utilize banks of cameras all imaging the same sample simultaneously to allow the collection of entire z-stacks and full volumetric imaging without the need for sample refocusing and/or increase the ability to resolve the location of molecular targets along the axial direction. These can increase imaging throughput and/or resolution in image-based approaches, e.g., for single-cell molecular profiling, or other applications.

Many biological questions require the measurement of genome-scale molecular profiles within single-cells in their native biological context. To meet this need, there has been a recent development of massively multiplexed molecular imaging techniques that can perform genome-scale profiling of the molecular properties of single cells. Such techniques can be used to target multiple distinct types of molecules within cells, including but not limited to genomic loci, mRNAs, and/or proteins.

Molecular specificity can be achieved by using probes that bind to the molecules of interest. For example, DNA oligonucleotides can be used to selectively label different nucleic acid species using sequences that are, in part, complementary to the sequences of the corresponding targets using techniques such as in situ hybridization (ISH) or fluorescence in situ hybridization (FISH). Similarly, nucleic acid aptamers or antibodies can be used to selectively label different proteins or proteins that have undergone selective post-translational modifications using techniques such as immunofluorescence or immunohistochemistry. The binding of these probes can be determined by labeling them with optically detectable moiety such as fluorophores and then imaging the sample with a variety of fluorescence microscopy methods. Distinct molecules can be determined simultaneously within the same sample by using optically distinguishable fluorophores. Alternatively, the number of distinct molecular species can be increased substantially beyond the number of distinguishable fluorophores through methods that repetitively stain and image the sample. In some cases, this process of repetitive staining and imaging of the same sample can be used to build combinatorial barcodes that allow the identification of a large number of molecular targets.

For example, MERFISH is a massively multiplexed form of single-molecule FISH. In one embodiment of MERFISH, unique binary barcodes can be assigned to each of the targeted nucleic acid species. These barcodes are then translated into encoding probes that target each of these nucleic acid species and which assign the corresponding barcodes to these molecules. In one implementation of this method, a unique oligonucleotide sequence—a readout sequence—is designed for each bit in the binary barcodes. The specific barcode for each targeted nucleic acid then determines the combination of readout sequences presence on the encoding probes targeted to the corresponding nucleic acid target. Specifically, if a barcode contains a “1” in a given bit, the corresponding encoding probes will contain that readout sequence; if it contains a “0” in that bit, then those probes will not contain that readout sequence. These probes are hybridized to the biological sample, e.g., using in situ hybridization methods. To determine which molecular targets are labeled with probes that contain a given readout sequence, the sample may then be hybridized with a fluorescently labeled readout probe complementary to that readout sequence. The sample is imaged, and the distribution of measured fluorescence may be used to determine the location of molecules with barcodes that contain a “1” in the bit corresponding to the targeted readout sequence. The fluorescence signal may then be removed from the sample, and the sample can be stained with a different fluorescently labeled readout probe complementary to a different readout sequence in order to determine the molecular targets that contain a “1” in that bit. The sample is imaged, fluorescence signal is removed, and this process is repeated until all bits in the barcodes have been measured. If multiple distinguishable fluorophores are associated with different readout probes, multiple readout probes can be stained simultaneously, and multi-color fluorescence imaging can be used to probe the value of multiple bits simultaneously.

It should be understood that similar approaches that employ more general barcoding approaches are also contemplated herein. For example, some barcoding approaches may involve trits or larger barcode alphabets. In some cases, some or all of the readout sequences can be assigned to all possible values of an alphabet, e.g., one unique readout sequence associated with a “1” value at a given bit and a different unique readout sequence associated with a “0” value at that bit. As yet another example, the absence of a signal can be associated with one of the values at a given barcode entry.

Such measurements can incur readout errors—in which the value of a given bit is not measured properly. These measurement errors can be problematic for multiplexed measurements as they can convert one barcode into another and/or lead to the misidentification of molecular targets. A variety of mechanisms can lead to these readout errors, such as the lack of binding of some of the nucleic acid probes or the misbinding of some probes to the wrong molecular targets. To overcome these errors, MERFISH can utilize binary or other barcoding schemes that can detect and/or correct bits or other elements that have been measured incorrectly. A wide range of error robust and correcting encoding schemes can be used in this approach, including Hamming codes, constant-weight Hamming codes, Golay codes, Turbo codes, Reed-Solomon codes, Reed-Solomon erasure codes, etc. In addition, encoding schemes that do not use binary barcodes can, nonetheless, be represented as binary barcodes. Thus, there is no loss in generality by assuming binary barcodes. The error detection and correcting capabilities of these barcodes arise from, for example, the Hamming distance that separates one valid barcode from another—e.g., the number of bits that must be corrupted to convert one barcode into another. Barcoding schemes such as the Hamming code are designed such that all valid barcodes are separated by a minimum Hamming distance. However, other barcoding schemes can also be generated by randomly selecting a suitably small subset of all possible barcodes such that the majority of generated barcodes are separated by a minimum desired Hamming distance and, thus, have the desired error detecting and correcting properties. Any coding scheme that utilizes barcodes separated by some minimum Hamming distance can be used in such applications to provide some degree of error detection and/or correction. As would be understood by those of ordinary skill in the art, the “Hamming distance” between two barcodes of equal length is the number of positions at which the corresponding symbols are different. In other words, the Hamming distance measures the minimum number of substitutions required to change one barcode into the other, or the minimum number of errors that could have transformed one barcode into the other.

A wide range of two-dimensional imaging modalities may be used with MERFISH and other image-based molecular profiling techniques in various embodiments. For example, wide-field imaging techniques such as epi-fluorescence microscopy or spinning disk confocal microscopy can be used. Similarly, two-dimensional scanning modalities may also be used in some cases, such as scanning point or line confocal. In addition, super-resolution imaging modalities may be used as well, including stochastic optical reconstruction microscopy (STORM), stimulated emission depletion microscopy (STED), or structured illumination microscopy. Sample preparation methods that physically expand the sample, such as expansion microscopy, can be combined with any of these imaging modalities to further improve resolution.

In some cases, these imaging modalities image a single optical plane at a given time and, thus, can produce two-dimensional (2D) images. However, there is often substantial benefit to reconstructing the molecular profiles of biological samples in three dimensions (3D). To reconstruct 3D distributions with such methods, the sample is often refocused by changing the distance between the imaging objective and the sample so that the imaging system is focused on a different axial plane within the sample. A 2D image is collected. The focus of the imaging system is adjusted, and another image is collected. By iterating this process, a series of 2D images each collected at a different focal plane can be used to reconstruct a 3D volume. In this process, a single camera or point or line detector can be used to reconstruct a 3D volume.

One drawback to these approaches to collecting information on a 3D volume is that they can be slow. First, images at different focal planes (or z positions) are collected in a serial fashion. Thus, to collect a stack of N difference images requires at least N times the exposure time for one image. Second, refocusing the system from one focal plane to another often requires some physical movement of system, either a movement of the objective lens or a movement of the sample, and there is a finite time required for the system to make such movements and properly settle at the new position.

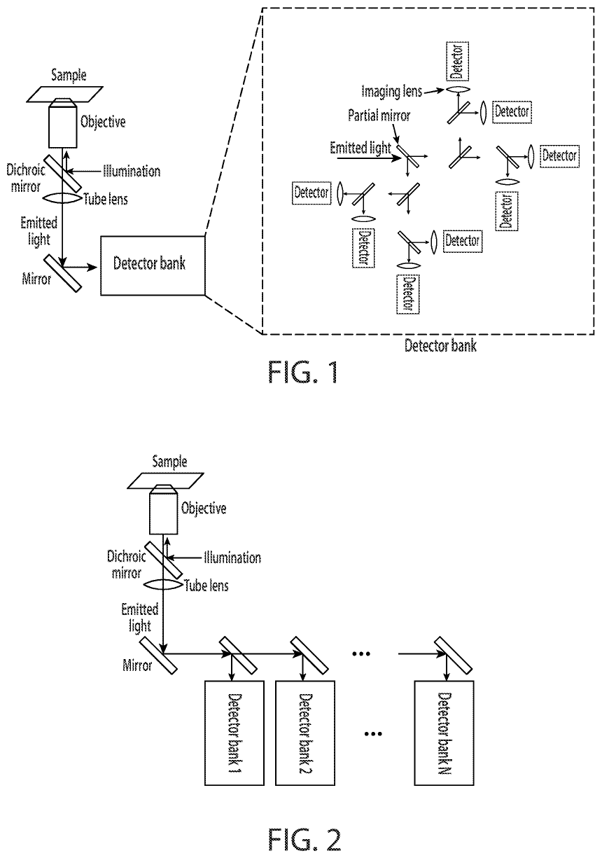

Accordingly, certain embodiments are generally directed to methods and apparatuses that allow widefield or point scanning images at multiple focal planes simultaneously. In some cases, these can be used to substantially improve the performance of highly multiplexed, image-based methods for molecular profiling within biological samples. In some embodiments, these approaches make use of one or more detector banks, as opposed to the typical optical instrument that utilizes one or two detectors to image the sample. An apparatus may comprise multiple detectors, e.g., no less than 2, 4, 8, 16, or 32 detectors, with each detector imaging a different focal plane at the same 2D location of the sample. To distribute light from this portion of the sample across the detectors within a single detector bank, partially reflecting mirrors (e.g. 50% mirrors) may be used. The optics within each detector bank may be configured such that each detector within a bank images the same 2D location in the sample but may image a different focal (z) position or focal offset at that plane. In some cases, a system may be capable of imaging all or a portion of the focal planes in the desired z-stack simultaneously. See, e.g., FIG. 1.

FIG. 1 is a schematic depiction of one embodiment illustrating an apparatus. In this non-limiting example, an illumination system provides light to excite fluorescence in a sample, and is coupled into an imaging objective via a dichroic mirror. Fluorescence for the sample is collected via a tube lens and directed into a detector bank. Within the detector bank, a partial mirror splits the emitted light into two optical paths. A series of additional partial mirrors separates each of these paths into four paths, and then each of these four paths is split into an additional two paths via another set of partial mirrors. Each of these mirrors can be adjusted to allow alignment of the system. The light from each path is then focused onto a detector associated with each path to form an image of the sample plane at that detector. By adjusting the relative distance between each detector and its corresponding imaging lens, the specific focal plane in the sample that is imaged by a given detector can be selected. By systematically aligning all lenses, each detector in the array can image a different focal plane in the sample.

In some embodiments, the detectors are cameras. The cameras may comprise a two-dimensional arrays of pixels, and may be capable of quantifying the amount of light impinging on each pixel. In other embodiments, the detectors may be point detectors, such as photodetectors or avalanche photodiodes. In still other embodiments, the detectors may be one dimensional arrays of pixels, e.g., line detectors. In yet other embodiments, a detector may be comprised of an additional bank of detectors that each detect some different property or parameter of the sample, e.g., the color of emitted light, the timing of emitted light, or the like.

In some embodiments, the parallel collection of multiple focal planes at a given location by such a system can decrease the time required to image a sample by a proportional amount. For example, if 8 cameras are used to image 8 different focal planes in a 8-image z-stack, the time required to collect this stack, as opposed to the standard methods of scanning the focus of the system and collecting these images in serial fashion, can be reduced, for example, at least 8-fold. In other embodiments, the use of such camera banks can decrease the time required to collect such a z-stack more than an amount proportional to the number of cameras, for example, if the system requires a sizeable amount of time to refocus between the collection of each image in the z-stack.

In some embodiments, the use of detector banks can decrease the time for collecting a z-stack of images, and in certain cases, by an amount that exceeds what one would expect simply by increasing the number of detectors being used (e.g., one might expect a 2-fold decrease in time by using 2 cameras). Such decreases in time may occur, for example, if the exposure time used in a detector bank is larger than the exposure time used in a comparable imaging experiment in which the z-stack is acquired via multiple rounds of refocusing of the sample and imaging with a single detector.

As an illustrative non-limiting example, consider a camera with a frame rate of 100 Hz, and a microscope system that requires 100 ms to reposition the objective at a new focal plane. Because the exposure time for each image (10 ms) is substantially faster than the time required to refocus the system, one must wait for the system to complete the refocusing process to collect an image. Thus, the time required to collect a z-stack comprised of 8 images will be the time required to expose all 8 images (80 ms) plus the time to perform the necessary 7 refocus events (700 ms). By contrast, using a plurality of cameras such as described here, refocusing is not necessary, and all 8 cameras within such a bank could be exposed simultaneously. Thus, the total time required to reconstruct the full z-stack using a single camera and refocusing (780 ms) could be reduced to just 10 ms using the camera bank system described here, e.g., comparable to the time for just a single camera.

Dividing light between multiple cameras may decrease the signal received by each camera, at least in certain embodiments. In some cases, if necessary, this decrease in optical signal can be overcome, for example, by amplifying the signal from individual molecular targets. Suitable methods include, but are not limited to, increasing the illumination intensity, using brighter fluorescent molecules, or by exposing the sample for longer durations. Methods that increase the number of fluorescent molecules bound to each molecular target can also be used in some instances. For example, multiple distinct FISH probes could be targeted to individual nucleic acids, or multiple fluorophores can be affixed to primary or secondary antibodies, etc.

In some embodiments, binding sites for fluorescently labeled probes may be replicated. Such methods include, but are not limited to, rolling circle amplification (RCA), hybridization chain reaction (HCR), clampFISH, branched DNA (bDNA) amplification, or the like. These and other methods can be used in some embodiments to increase the signal from individual molecular targets, e.g., such that a plurality of cameras may be used.

In another embodiment, multiple camera banks are utilized with the light collected from the objective split via color using optics such as dichroics such that individual color channels are directed to individual camera banks (FIG. 2). Within each camera bank, individual cameras can be focused on different focal planes, and individual camera banks can be used to image the same sample volume, but in different color channels. In some cases, full or partial z-stacks can be collected in multiple color channels in parallel allowing a multi-color z-stack to be collected in the time required for the exposure of a single camera.

In some embodiments, the time required to image samples may be reduced. For example, collecting a full z-stack of images, e.g., using a plurality of different colors, would mean that multiple readout probes could be measured in a round of staining of imaging, rather than requiring a round of imaging for each bit. A non-limiting example of such an approach is shown in Example 2.

In some embodiments, the camera banks can be designed so that the axial distance separating each imaged optical plane is uniform, while in other embodiments the spacing between imaged planes may be not uniform, or different. In certain embodiments, the camera banks can be designed so that the focal plane imaged by each camera can be rapidly changed, independently of the other planes imaged by cameras within the same bank. The ability to change the spacing between the imaged focal planes within a camera bank may allow the optimization of the z-stack properties for a given measurement in certain cases. For example, in measurements of molecular profiles within cells, it may not be necessary to image the same molecule within closely spaced optical planes, but rather, to separate the optical planes by a distance equal to or larger than the axial extent of the point spread function of the imaging system so that signal from a single molecule appears in only one optical plane. In this imaging modality, each image would represent a statistically distinct measure of the molecular profile within individual cells. By contrast, in other applications, it may be beneficial to decrease the spacing between imaged optical planes such that the spacing is equal to or smaller than the axial extent of the optical point spread function and, thus, the signal from a single molecule will appear on multiple cameras within a bank.

There are multiple ways in which data from multi-focal camera banks can be analyzed to reconstruct properties of the imaged systems, in accordance with some embodiments of the disclosure. For example, as mentioned above, independent planes may be analyzed independently and may be used to provide statistically independent measures of the expression profiles within regions of the sample, e.g. individual cells. In some cases, information from multiple planes may be used collectively in the analysis of the z-stack. In other words, an image from one z-plane can be used in the analysis of an image from a different z-plane. For example, in certain embodiments, images collected from a single camera bank may be used in combination with optical deconvolution algorithms to reconstruct a 3D image of the distribution of molecules within the imaged volume of the sample, e.g., at a resolution higher than the optical resolution of the system. In addition, such images could be analyzed, in some embodiments, with multi-plane localization algorithms to allow the 3D location of single molecules to be determined with a resolution below the diffraction limit. In addition, optical reconstruction techniques can be used in some embodiments to combine the in-focus intensity information in one plane with the out-of-focus light collected in a second plane to determine the intensity and phase of the emitted light from the sample and, thus, reconstruct the full 3D distribution of the fluorescent signal in the imaged volume.

In some embodiments, for systems that employ multiple camera banks each dedicated to a separate color channel, such analyses can be performed independently for each of these colors channels. However, it is also possible in certain cases to combine information from images collected from different color banks. For example, if individual camera banks collect light from different portions of the emission spectra of distinct fluorophores, methods such as spectral demixing can be used to determine the distribution of two or more fluorophores with distinct but overlapping emission spectra.

In yet another embodiment, camera-based wide-field imaging could be replaced with banks of point detectors. In some embodiments, a set of illumination spots displaced along the axial position throughout the sample can be mapped to each point-detector in a bank of point detectors. By simultaneously scanning the illumination spots and the position in the sample at which the point detectors are collecting light, a z-stack of scanned images may be constructed, in which the different z-planes are imaged simultaneously. This can also be used with other detectors, such as line illumination and line detectors. By appropriately masking the emitted light, such measurements can be performed in a confocal modality. In various embodiments, any type of light sensitive detector could be used in such banks to allow a wide range of imaging modalities, including wide-field and scanning, to simultaneously characterize multiple axial positions in the sample.

In yet another embodiment, the detectors within a bank and, potentially, across multiple banks can be synchronized via electronics so that each detector begins and finishes its acquisition at the same time. In some embodiments, defined temporal delays can be set between the start of the acquisition for different detectors within a bank. Similarly, the acquisition between camera banks can begin at the same time or occur at specific time delays such that the acquisition duration is either completely, partially, or not at all overlapping, etc., at least in some embodiments. Multiple methods by which this type of acquisition timing can be controlled exist. In some embodiments, the acquisition timing of each detector within a bank can be set by reference to a timing signal produced by one “main” detector in the bank. In some cases, the timing of all detectors within a bank may be set by a timing signal produced by a camera in a separate bank. In yet other embodiments, the timing of all cameras within a bank may be set by a timing signal produced by an external source such as a signal generator or a computer. In some implements, external timing of the cameras may itself be triggered when some or all cameras indicate they are ready to receive a trigger.

In some embodiments, the data produced by each detector bank can be collected via a signal acquisition system, e.g., a computer. In some embodiments, the data produced by individual detectors within a bank can each be acquired by a dedicated signal acquisition system, i.e. a computer.

In some embodiments, the acquired data can be stored on storage systems, e.g. hard drives, that are shared by all detectors within a bank. In certain embodiments, the acquired data may be stored on storage systems that are unique to each detector. In some cases, these data can be aggregated, combined, and/or moved to separate storage by computing resources shared by all detectors within a bank, or within the system, or via computing resources unique to each detector and storage system, etc. In some implementations, single-board computers or other computers associated with cameras in the bank may contain internal storage systems, such as solid-state drives, to allow images to stream from one or more cameras, in real-time, to this internal storage. This storage may serve as temporary storage to stage data prior to transfer to other external system.

In some embodiments, the data can be streamed, for example, in real-time, to off-site storage systems using systems such as Ethernet communication, e.g. streaming to cloud storage, or other techniques for data transfer known to those of ordinary skill in the art.

In some embodiments, the collected data can be processed, for example, in real time. For example, in certain embodiments, computing resources that are shared between all detectors within a bank or which are unique to each detector within a bank can be used to process the data. Such processing can include, but is not limited to, computational tasks such as image deconvolution, low-pass filtering, high-pass filtering, feature identification, etc. In some embodiments, feature identification involves the identification of point- or spot-like features within images collected by these detectors, e.g. fitting of point-spread functions to determine the centroid of a fluorescent emitter. In some embodiments, data from multiple detectors in a bank representing information from multiple focal planes may be analyzed together. For example, in the context of image deconvolution, 3D deconvolution algorithms can be applied to an entire z-stack to reconstruct the 3D distribution at higher resolution. As another non-limiting example, spot fitting algorithms can be used to identify the 3D centroids of individual fluorescent emitters in such z-stacks.

In some embodiments, all or a portion of image analysis can be performed via dedicated functionality within the detectors of the detector bank. For example, individual cameras or other detectors may contain functionality that allows collected images to be deconvolved, to have specific regions of interest identified, to have features such as fluorescent spots identified, etc. In some cases, only these analysis results may be saved and/or sent to a computer, etc.

In some embodiments, the acquire images from the sample can be discarded after such analysis is performed.

In some embodiments, a single computer running a single master control program can be used for the coordination of data collection from all detectors in all detector banks attached to a single microscope. In certain cases, each detector bank can be controlled via its own computer running a separate program, and these computers may be, in some cases, controlled via a master computer running software that communicates with and coordinates the programs running on the computer associated with each detector bank. In some embodiments, each detector within a bank can be controlled by a computer associated with that camera running a control program. In certain embodiments, these computers may in turn be controlled by a master computer associated with each detector bank, which may run a master control program responsible for communication with all computers on the bank.

Furthermore, in some embodiments, sets of cameras, e.g. 2, 3, 4, or more cameras can be controlled by a single computer, e.g., such that the camera bank is controlled by more than one computer but fewer computers than the total number of cameras. In addition, in some embodiments, a small group of single-board computers that are responsible for controlling the camera bank systems may, in turn, be controlled by a separate computer, such as a tower or personal computer, e.g., that is responsible for controlling other aspects of the imaging system. Single-board computers may contain multiple CPUs or GPUs, which may facilitate real-time processing of data from individual cameras or sets of cameras within camera banks. Communication between these computers can be performed, for example, via various speeds, e.g., 10 Gb or 1 Gb, e.g., using ethernet or wireless communication methods. In addition, communication could be performed via USB, serial, digital TTL, or the like.

In some embodiments, the analysis of the images may be performed, at least in part, via machine learning approaches. For example, in one embodiment neural networks may be trained with a subset of data collected on individual detectors or all detectors in a detector bank using a suitable sample, and then such networks could be used to analyze the data collected by individual detectors within a detector bank, or to perform joint analysis on all detectors within the bank, or the like. In some embodiments, such analysis could be used instead of precise calibration of the spacing of the optical planes imaged by each detector in the detector bank. In some embodiments, such analysis can be used to increase the speed by which features of interest are identified for such images, e.g., relative to other analysis methods. In certain cases, such methods can be used to increase the effective resolution of the image reconstructed from all detectors in the detector bank. In some embodiments, such methods can be used to jointly analyze data collected from multiple detector banks imaging different portions of a sample, such as, for example, different colors of fluorescent emitter. Such methods can be used, for example, to distinguish the emission of more fluorophores than the number of detector banks used to image the sample.

A wide range of machine learning approaches are available, including but not limited to support vector machines, linear and non-linear regression, and different forms of neural networks. In addition, there are a wide range of technologies for implementing such analyses, including methods for running this analysis in real-time, i.e., prior to the saving of images. These methods include, but are not limited to, field-programmable gate arrays (FPGAs), graphic processer units (GPUs), CPUs, or the like.

In some embodiments, any of the optical apparatuses discussed herein can be used, for example, in a multiplexed single-molecule RNA imaging measurement, multiplexed immunofluorescence imaging, multiplexed genomic measurements, or in multi-modal measurements that combine two or more of the above techniques and/or additional techniques. In certain cases, optical apparatuses such as those described herein can be used to follow the movement of objects in 3D space in real time, e.g., tracking of fluorescently labeled molecules within single cells.

Certain embodiments are directed to systems and methods to align and/or maintain the alignment of such systems. For example, a fixed set of fiducial points, e.g. fluorescent beads embedded in a hydrogel or placed on the surface of a sample, can be scanned along the optical axis, and the relative scan position at which a given fiducial point is in focus on each detector can be used to determine the relative offset between the focal planes imaged by each detector, and/or to adjust the imaged focal position for each detector to match a pre-determined, desired focal plane.

In some embodiments, a reference light source, such as a collimated laser, is directed through the detector bank, for example, to align the system. In some embodiments, the focusing or imaging lens associated with each detector can be scanned to determine the position of best focus for this alignment source. Then the imaging lens may be moved a predetermined offset from the position of this focus. In some embodiments, in which some detectors share an imaging lens, a comparable alignment may be performed by moving each detector along the optical axis. Similarly, the alignment of each detector in the bank may be set by the image of this alignment source on each detector, e.g. the measured width of the spot formed by this alignment source. Similarly, each mirror, lens, or detector may be moved to align the image constructed by each element in the detector array so that the 2D positions of these images are in alignment. For example, with cameras, such alignment may register the position of corresponding pixels in two different cameras in the detector bank to the same location in the sample. Such alignment can be performed with an accuracy better than, for example, 5 nm, 10 nm, 20 nm, 30 nm, 50 nm, 100 nm, 200 nm, 500 nm, or 1000 nm. Similarly, the alignment of the desired focal plane associated with each detector can be done with an accuracy of better than, for example, 10 nm, 25 nm, 100 nm, 200 nm, 500 nm, and 1000 nm.

The present disclosure is not limited to only the above examples. Other embodiments are also possible. Accordingly, more generally, various aspects of the disclosure are directed to various systems and methods for multi-focal imaging.

For example, certain embodiments of the disclosure are generally directed to systems and methods of using a plurality of detectors that are focused on various focal planes, e.g., in a sample. Examples of detectors include, but are not limited to, cameras, photodetectors, photodiodes, or the like. Examples of cameras include, but are not limited to CCD cameras, CMOS cameras, or optical cameras. In some cases, the camera includes a two-dimensional arrays of pixels, which may be used to quantify the amount of light reaching each pixel. Many such cameras are available commercially, and often at relatively low cost. Other examples of detectors include line detectors or point detectors, such as photodetectors or photodiodes. The detectors may independently the same or different. For example, in some cases, the detectors may detect various properties of a sample, e.g., the color of emitted light, the timing of emitted light, or the like.

The detectors may each be focused on a different focal plane, and/or 2 or more of the detectors may be focused on the same plane. For example, in some embodiments, the focal planes are positioned to be relatively close to each other. For example, a focal plane may be positioned at no more than 1000 nm from the closest neighboring focal plane, and in some cases, no more than 750 nm, no more than 600 nm, no more than 500 nm, no more than 300 nm, no more than 200 nm, no more than 100 nm, no more than 75 nm, no more than 60 nm, no more than 50 nm, no more than 40 nm, no more than 30 nm, no more than 20 nm, or no more than 10 nm from the closest neighboring focal plane. The focal planes may also be distributed evenly (e.g., there is approximately the same distance between each of the neighboring focal planes), or unevenly in some embodiments. In some cases, the focal planes are substantially parallel to each other.

In certain embodiments, the use of a plurality of focal planes may allow the z (axial) positions of entities within the sample to be determined at relatively high resolutions, e.g., better than 1000 nm, 750 nm, 600 nm, 500 nm, 300 nm, 200 nm, 100 nm, 75 nm, 60 nm, 50 nm, 40 nm, 30 nm, 20 nm, or 10 nm. In some cases, the resolution may be related to the separation of the focal planes.

The focal plane may be defined, at least in part, by imaging lenses positioned to individually affect some or all of the detectors, e.g., as is shown in FIG. 1. Thus, for example, each of the detectors may be affected by a respective imaging lens, which may be used to control the focal plane of that particular detector. However, it should be understood that other optical elements, such as lenses, mirrors, beamsplitters, etc. may also be present, e.g., within optical paths leading from the samples to the detectors. In addition, in certain cases, not all of the detectors may be affected by an individual imaging lens.

In some cases, the imaging lenses may be individually focused, e.g., by independently moving the lens to a position that allows an image to be focused on the detector, or by other focusing techniques. In some cases, a plurality of imaging lenses may be controlled to focus on different imaging planes by focusing some or all of the detectors using the imaging lenses onto a sample (e.g., onto a test image), then moving some or all of the lenses by various amounts to alter the focal plane of that lens. For instance, at least some of the lenses may be moved by an offset (or a multiplicity of offsets) to cause the focal plane to also be offset. For example, moving a lens by an offset may cause the focal plane to also move by an offset, and the offset of the lens may be proportional to the offset of the focal plane. In some cases, the offset is a fixed amount. Thus, for example, a first lens may be moved by a first offset and a second lens may be moved by a second offset (for example, equal to 2 times the first offset), etc. to cause the focal planes of the imaging lenses to be different. In addition, in certain embodiments, focusing can be performed by moving the detectors themselves along the optical axis, e.g., without necessarily moving lenses.

In some cases, the arrangement of cameras and lenses may effectively interlink cameras associated with different aspects of imaging. For example, 16 cameras could be used to image 2 colors in 8 image planes by creating two 8-camera banks that are optically separated by a dichroic beamsplitter that separates light in the two channels and directs each color band to each of the two 8-camera banks. As another example, a series of non-dichroic beam splitters that separate light independent of color could be used to divide collected light into 8 separate optical paths that each contain a dichroic beam splitter that separately directs two different color bands of light to two cameras associated with each of the 8 optical paths. In some cases, by placing the optical elements at different locations, a wide range of different camera bank organizations could be implemented that still result in two-color, 8-optical-plane images being simultaneously collected, or other arrangements such as those discussed herein. In addition, it will be recognized that generalizations of this approach can easily be extended to more than two colors (e.g., 3, 4, 5, 6, etc. colors), different numbers of optical planes (e.g., 4 planes, 6 planes, 10 planes, 12 planes, 14 planes, 16, planes, etc.), and/or other properties of light that can be discriminated, such as polarization.

In some cases, relatively large numbers of detectors may be used. For example, light from a sample may be split into different pathways (e.g., using beamsplitters) to channel light into the various detectors. Thus, for example, there may be 2ⁿ detectors present, based on the number of beamsplitters n that are used. For instance, if 2 beamsplitters are used, there may be 2²=4 detectors; if 3 are used, there may be 2³=8 detectors, etc. As other examples, there may be 4, 5, 6, 7, 8 or more beamsplitters used, e.g., corresponding to at least 16, 32, 64, 128, etc., or more detectors being used.

The detectors may independently be the same or different. For example, the detectors may be turned to capture substantially the same frequencies, and/or some detectors or groups of detectors may be set to capture different frequencies, e.g., to capture different “colors” from a sample. For example, there may be a first group or bank of cameras for capturing a first color and a second group or bank of cameras for capturing a second color.

In addition, the detectors may independently be set to capture images at the same time, or at different times. For example, there may be a first group or bank of cameras for capturing an image at a first time, and a second group or bank of cameras for capturing an image at a second time. If enough detectors are used, “movies” of a sample may also be created in certain embodiments. In addition, techniques such as any of these described herein may be combined together, for example, for capturing “movies” of a sample in two or more colors.

In some cases, the amount of light arriving at each detector is affected by the number of beamsplitters the light passes through. Thus, for example, the light reaching a detector after having passed through 3 beamsplitters may be ⅛ (½³) of the original intensity. Thus, in certain embodiments, the light from the sample may be amplified, for example, signaling entities on the sample may be amplified to increase the amount of light produced, such that the light reaching each of the detectors is of sufficient intensity for analysis. Examples of signaling entities include, but are not limited to, those disclosed herein, and some non-limiting methods of amplifying signaling entities are provided below and in U.S. Pat. Apl. Ser. No. 62/779,333, incorporated herein by reference in its entirety.

Certain aspects thus are directed to determining a sample, which may include a cell culture, a suspension of cells, a biological tissue, a biopsy, an organism, or the like. The sample can also be cell-free but nevertheless contain nucleic acids in some cases. If the sample contains a cell, the cell may be a human cell, or any other suitable cell, e.g., a mammalian cell, a fish cell, an insect cell, a plant cell, or the like. More than one cell may be present in some cases.

Within the sample, the targets to be determined can include nucleic acids, proteins, or the like. Nucleic acids to be determined may include, for example, DNA (for example, genomic DNA), RNA, or other nucleic acids that are present within a cell (or other sample). The nucleic acids may be endogenous to the cell, or added to the cell. For instance, the nucleic acid may be viral, or artificially created. In some cases, the nucleic acid to be determined may be expressed by the cell. The nucleic acid is RNA in some embodiments. The RNA may be coding and/or non-coding RNA. For example, the RNA may encode a protein. Non-limiting examples of RNA that may be studied within the cell include mRNA, siRNA, rRNA, miRNA, tRNA, lncRNA, snoRNAs, snRNAs, exRNAs, piRNAs, or the like.

In some cases, a significant portion of the nucleic acid within the cell may be studied. For instance, in some cases, enough of the RNA present within a cell may be determined so as to produce a partial or complete transcriptome of the cell. In some cases, at least 4 types of mRNAs are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 types of mRNAs may be determined within a cell.

In some cases, the transcriptome of a cell may be determined. It should be understood that the transcriptome generally encompasses all RNA molecules produced within a cell, not just mRNA. Thus, for instance, the transcriptome may also include rRNA, tRNA, siRNA, etc. in certain instances. In some embodiments, at least 5%, at least 10%, at least 15%, at least 20%, at least 25%, at least 30%, at least 40%, at least 50%, at least 60%, at least 70%, at least 80%, at least 90%, or 100% of the transcriptome of a cell may be determined.

In some embodiments, other targets to be determined can include targets that are linked to nucleic acids, proteins, or the like. For instance, in one set of embodiments, a binding entity able to recognize a target may be conjugated to a nucleic acid probe. The binding entity may be any entity that can recognize a target, e.g., specifically or non-specifically. Non-limiting examples include enzymes, antibodies, receptors, complementary nucleic acid strands, aptamers, or the like. For example, an oligonucleotide-linked antibody may be used to determine a target. The target may bind to the oligonucleotide-linked antibody, and the oligonucleotides determined as discussed herein.

The determination of targets, such as nucleic acids within the cell or other sample, may be qualitative and/or quantitative. In addition, the determination may also be spatial, e.g., the position of the nucleic acids, or other targets, within the cell or other sample may be determined in two or three dimensions. In some embodiments, the positions, number, and/or concentrations of nucleic acids, or other targets, within the cell or other sample may be determined.

In some cases, a significant portion of the genome of a cell may be determined. The determined genomic segments may be continuous or interspersed on the genome. For example, in some cases, at least 4 genomic segments are determined within a cell, and in some cases, at least 3, at least 4, at least 7, at least 8, at least 12, at least 14, at least 15, at least 16, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 genomic segments may be determined within a cell.

In some cases, the entire genome of a cell may be determined. It should be understood that the genome generally encompasses all DNA molecules produced within a cell, not just chromosome DNA. Thus, for instance, the genome may also include, in some cases, mitochondria DNA, chloroplast DNA, plasmid DNA, etc., e.g., in addition to (or instead of) chromosome DNA. In some embodiments, at least about 5%, at least about 10%, at least about 15%, at least about 20%, at least about 25%, at least about 30%, at least about 40%, at least about 50%, at least about 60%, at least about 70%, at least about 80%, at least about 90%, or 100% of the genome of a cell may be determined.

As discussed herein, a variety of nucleic acid probes may be used to determine one or more targets within a cell or other sample. The probes may comprise nucleic acids (or entities that can hybridize to a nucleic acid, e.g., specifically) such as DNA, RNA, LNA (locked nucleic acids), PNA (peptide nucleic acids), and/or combinations thereof. In some cases, additional components may also be present within the nucleic acid probes, e.g., as discussed below. In addition, any suitable method may be used to introduce nucleic acid probes into a cell.

For example, in some embodiments, the cell is fixed prior to introducing the nucleic acid probes, e.g., to preserve the positions of the nucleic acids or other targets within the cell. Techniques for fixing cells are known to those of ordinary skill in the art. As non-limiting examples, a cell may be fixed using chemicals such as formaldehyde, paraformaldehyde, glutaraldehyde, ethanol, methanol, acetone, acetic acid, or the like. In one embodiment, a cell may be fixed using HEPES-glutamic acid buffer-mediated organic solvent (HOPE).

The nucleic acid probes may be introduced into the cell (or other sample) using any suitable method. In some cases, the cell may be sufficiently permeabilized such that the nucleic acid probes may be introduced into the cell by flowing a fluid containing the nucleic acid probes around the cells. In some cases, the cells may be sufficiently permeabilized as part of a fixation process; in other embodiments, cells may be permeabilized by exposure to certain chemicals such as ethanol, methanol, Triton, or the like. In addition, in some embodiments, techniques such as electroporation or microinjection may be used to introduce nucleic acid probes into a cell or other sample.

Certain aspects are thus generally directed to nucleic acid probes that are introduced into a cell (or other sample). The probes may comprise any of a variety of entities that can hybridize to a nucleic acid, typically by Watson-Crick base pairing, such as DNA, RNA, LNA, PNA, etc., depending on the application. The nucleic acid probe typically contains a target sequence that is able to bind to at least a portion of a target, e.g., a target nucleic acid. In some cases, the binding may be specific binding (e.g., via complementary binding). When introduced into a cell or other system, the target sequence may be able to bind to a specific target (e.g., an mRNA, or other nucleic acids as discussed herein). The nucleic acid probe may also contain one or more read sequences, as discussed below.

In some cases, more than one type of nucleic acid probe may be applied to a sample, e.g., sequentially or simultaneously. For example, there may be at least 2, at least 5, at least 10, at least 25, at least 50, at least 75, at least 100, at least 300, at least 1,000, at least 3,000, at least 10,000, or at least 30,000 distinguishable nucleic acid probes that are applied to a sample. In some cases, the nucleic acid probes may be added sequentially. However, in some cases, more than one nucleic acid probe may be added simultaneously.

The nucleic acid probe may include one or more target sequences, which may be positioned anywhere within the nucleic acid probe. The target sequence may contain a region that is substantially complementary to a portion of a target, e.g., a target nucleic acid. For instance, in some cases, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary, e.g., to produce specific binding. Typically, complementarity is determined on the basis of Watson-Crick nucleotide base pairing.

In some cases, the target sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the target sequence may be no more than 500, no more than 450, no more than 400, no more than 350, no more than 300, no more than 250, no more than 200, no more than 175, no more than 150,no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the target sequence may have a length of between 10 and 30 nucleotides, between 20 and 40 nucleotides, between 5 and 50nucleotides, between 10 and 200 nucleotides, or between 25 and 35 nucleotides, between 10 and 300 nucleotides, etc.

The target sequence of a nucleic acid probe may be determined with reference to a target suspected of being present within a cell or other sample. For example, a target nucleic acid to a protein may be determined using the protein's sequence, e.g., by determining the nucleic acids that are expressed to form the protein. In some cases, only a portion of the nucleic acids encoding the protein are used, e.g., having the lengths as discussed above. In addition, in some cases, more than one target sequence that can be used to identify a particular target may be used. For instance, multiple probes can be used, sequentially and/or simultaneously, that can bind to or hybridize to the same or different regions of the same target. Hybridization typically refers to an annealing process by which complementary single-stranded nucleic acids associate through Watson-Crick nucleotide base pairing (e.g., hydrogen bonding, guanine-cytosine and adenine-thymine) to form double-stranded nucleic acid.

In some embodiments, a nucleic acid probe may also comprise one or more “read” sequences. The read sequences may be used, to identify the nucleic acid probe, e.g., through association with signaling entities, as discussed below. In some embodiments, the nucleic acid probe may comprise 1, 2, 3, 4, 5, 6, 7, 8, 9, 10, 11, 12, 13, 14, 15, 16 or more, 20 or more, 24 or more, 32 or more, 40 or more, 48 or more, 50 or more, 64 or more, 75 or more, 100 or more, 128 or more read sequences. The read sequences may be positioned anywhere within the nucleic acid probe. If more than one read sequence is present, the read sequences may be positioned next to each other, and/or interspersed with other sequences.

The read sequences may be of any length. If more than one read sequence is used, the read sequences may independently have the same or different lengths. For instance, the read sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

The read sequence may be arbitrary or random in some embodiments. In certain cases, the read sequences are chosen so as to reduce or minimize homology with other components of the cell or other sample, e.g., such that the read sequences do not themselves bind to or hybridize with other nucleic acids suspected of being within the cell or other sample. In some cases, the homology may be less than 10%, less than 8%, less than 7%, less than 6%, less than 5%, less than 4%, less than 3%, less than 2%, or less than 1%. In some cases, there may be a homology of less than 20 basepairs, less than 18 basepairs, less than 15 basepairs, less than 14 basepairs, less than 13 basepairs, less than 12 basepairs, less than 11 basepairs, or less than 10 basepairs. In some cases, such basepairs are sequential.

In one set of embodiments, a population of nucleic acid probes may contain a certain number of read sequences, which may be less than the number of targets of the nucleic acid probes in some cases. Those of ordinary skill in the art will be aware that if there is one signaling entity and n read sequences, then in general 2n−1 different nucleic acid targets may be uniquely identified. However, not all possible combinations need be used. For instance, a population of nucleic acid probes may target 12 different nucleic acid sequences, yet contain no more than 8 read sequences. As another example, a population of nucleic acids may target 140 different nucleic acid species, yet contain no more than 16 read sequences. Different nucleic acid sequence targets may be separately identified by using different combinations of read sequences within each probe. For instance, each probe may contain 1, 2, 3, 4, 5, 6, 7, 8, 9, 10, 11, 12, 13, 14, 15, 16, etc. or more read sequences. In some cases, a population of nucleic acid probes may each contain the same number of read sequences, although in other cases, there may be different numbers of read sequences present on the various probes.

As a non-limiting example, a first nucleic acid probe may contain a first target sequence, a first read sequence, and a second read sequence, while a second, different nucleic acid probe may contain a second target sequence, the same first read sequence, but a third read sequence instead of the second read sequence. Such probes may thereby be distinguished by determining the various read sequences present or associated with a given probe or location, as discussed herein. For example, the probes can be sequentially identified and encoded using “codewords,” as discussed below. Optionally, the codewords may also be subjected to error detection and/or correction.

In addition, the population of nucleic acid probes (and their corresponding, complimentary sites on the encoding probes), in certain embodiments, may be made using only 2 or only 3 of the 4 naturally occurring nucleotide bases, such as leaving out all the “G”s or leaving out all of the “C”s within the population of probes. Sequences lacking either “G”s or “C”s may form very little secondary structure in certain embodiments, and can contribute to more uniform, faster hybridization. Thus, in some cases, the nucleic acid probes may contain only A, T, and G; only A, T, and C; only A, C, and G; or only T, C, and G.

In one aspect, the read sequences on the nucleic acid probes may be able to bind (e.g., specifically) to corresponding recognition sequences on the primary amplifier nucleic acids. Thus, when a nucleic acid probe recognizes a target within a biological sample, e.g., a DNA or RNA target, the primary amplifier nucleic acid are also able to associate with the target via the nucleic acid probe, with interactions between the read sequences of the nucleic acid probes and corresponding recognition sequences on the primary amplifier nucleic acids, e.g., complementary binding. For instance, the recognition sequence may be able to recognize a target read sequence, but not substantially recognize or bind to other, non-target read sequence. The primary amplifier nucleic acids may also comprise any of a variety of entities able to hybridize a nucleic acid, e.g., DNA, RNA, LNA, and/or PNA, etc., depending on the application. For instance, such entities may form some or all of the recognition sequence.

In some cases, the recognition sequence may be substantially complementary to the target read sequence. In some cases, the sequences may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary. Typically, complementarity is determined on the basis of Watson-Crick nucleotide base pairing. The structures of the target read sequence may include those previously described.

In some cases, the recognition sequence may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the recognition sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

In some embodiments, a primary amplifier nucleic acid may also comprise one or more read sequences able to bind to secondary amplifier nucleic acids, as discussed below. For example, a primary amplifier nucleic acid may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more read sequences. The read sequences may be positioned anywhere within the primary amplifier nucleic acid. If more than one read sequence is present, the read sequence may be positioned next to each other, and/or interspersed with other sequences. In one embodiment, the primary amplifier nucleic acid comprises a recognition sequence at a first end and a plurality of read sequences at a second end.

In some cases, a read sequence within the primary amplifier nucleic acid may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence may have a length of between 10 and 20 nucleotides, between 10 and 30 nucleotides, between 20 and 40 nucleotides, between 5 and 50 nucleotides, between 10 and 200 nucleotides, or between 25 and 35 nucleotides, between 10 and 300 nucleotides, etc.

There may be any number of read sequences within a primary amplifier nucleic acid. For example, there may be 1, 2, 3, 4, 5, 6, 7, 8, 9, 10, 11, 12, 13, 14, 15, 16, 17, 18, 19, 20, or more read sequences present within a primary amplifier nucleic acid. If more than one read sequence is present within a primary amplifier nucleic acid, the read sequences may be the same or different. In some cases, for example, the read sequences may all be identical.

In some embodiments, the population of primary amplifier nucleic acids may be made using only 2 or only 3 of the 4 naturally occurring nucleotide bases, such as leaving out all the “G”s or leaving out all of the “C”s within the population of nucleic acids. Sequences lacking either “G”s or “C”s may form very little secondary structure in certain embodiments, and can contribute to more uniform, faster hybridization. Thus, in some cases, the primary amplifier nucleic acids may contain only A, T, and G; only A, T, and C; only A, C, and G; or only T, C, and G.

In some cases, more than one type of primary amplifier nucleic acid may be applied to a sample, e.g., sequentially or simultaneously. For example, there may be at least 2, at least 5, at least 10, at least 25, at least 50, at least 75, at least 100, at least 300, at least 1,000, at least 3,000, at least 10,000, or at least 30,000 distinguishable primary amplifier nucleic acids that are applied to a sample. In some cases, the primary amplifier nucleic acids may be added sequentially. However, in some cases, more than one primary amplifier nucleic acid may be added simultaneously.

In one set of embodiments, the read sequences on the primary amplifier nucleic acids may be able to bind (e.g., specifically) to corresponding recognition sequences on the secondary amplifier nucleic acids. Thus, when a nucleic acid probe recognizes a target within a biological sample, e.g., a DNA or RNA target, the secondary amplifier nucleic acids are also able to associate with the target, via the primary amplifier nucleic acids, with interactions between the read sequences of the primary amplifier nucleic acids and corresponding recognition sequences on the secondary amplifier nucleic acids, e.g., complementary binding. For instance, the recognition sequence on a secondary amplifier nucleic acid may be able to recognize a read sequence on a primary amplifier nucleic acid, but not substantially recognize or bind to other, non-target read sequence. The secondary amplifier nucleic acids may also comprise any of a variety of entities able to hybridize a nucleic acid, e.g., DNA, RNA, LNA, and/or PNA, etc., depending on the application. For instance, such entities may form some or all of the recognition sequence.

In some cases, the recognition sequence on the secondary amplifier nucleic acid may be substantially complementary to a read sequence on a primary amplifier nucleic acid. In some cases, the sequences may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

In some cases, the recognition sequence on the secondary amplifier nucleic acid may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the recognition sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the recognition sequence may have a length of between 10 and 30 nucleotides, between 20 and 40 nucleotides, between 5 and 50 nucleotides, between 10 and 200 nucleotides, or between 25 and 35 nucleotides, between 10 and 300 nucleotides, etc.

In some embodiments, a secondary amplifier nucleic acid may also comprise one or more read sequences able to bind to a signaling entity, as discussed herein. For example, a secondary amplifier nucleic acid may comprise 1, 2, 3, 4, 5, 6, 7, 8, 9, 10, 11, 12, 13, 14, 15, 16 or more, 20 or more, 32 or more, 40 or more, 50 or more, 64 or more, 75 or more, 100 or more, 128 or more read sequences able to bind to a signaling entity. The read sequences may be positioned anywhere within the secondary amplifier nucleic acid. If more than one read sequences is present, the read sequences may be positioned next to each other, and/or interspersed with other sequences. In one embodiment, the secondary amplifier nucleic acid comprises a recognition sequence at a first end and a plurality of read sequences at a second end. This structure may also be the same or different than the structure of the primary amplifier nucleic acid.

In some cases, the read sequence within the secondary amplifier nucleic acid may be at least 5, at least 10, at least 15, at least 20, at least 25, at least 30, at least 35, at least 40, at least 50, at least 60, at least 65, at least 75, at least 100, at least 125, at least 150, at least 175, at least 200, at least 250, at least 300, at least 350, at least 400, or at least 450 nucleotides in length. In some cases, the read sequence may be no more than 500, no more than 450, no more than 400, no more than 350, no more than 300, no more than 250, no more than 200, no more than 175, no more than 150, no more than 125, no more than 100, be no more than 75, no more than 60, no more than 65, no more than 60, no more than 55, no more than 50, no more than 45, no more than 40, no more than 35, no more than 30, no more than 20, or no more than 10 nucleotides in length. Combinations of any of these are also possible, e.g., the read sequence within the secondary amplifier nucleic acid may have a length of between 10 and 20 nucleotides, between 10 and 30 nucleotides, between 20 and 40 nucleotides, between 5 and 50 nucleotides, between 10 and 200 nucleotides, or between 25 and 35 nucleotides, between 10 and 300 nucleotides, etc.

There may be any number of read sequences within a secondary amplifier nucleic acid. For example, there may be 1, 2, 3, 4, 5, 6, 7, 8, 9, 10, 11, 12, 13, 14, 15, 16, 17, 18, 19, 20, or more read sequences present within a secondary amplifier nucleic acid. If more than one read sequence is present within a secondary amplifier nucleic acid, the read sequences may be the same or different. In some cases, for example, the read sequences may all be identical. In addition, there may independently be the same or different numbers of read sequences in the primary and in the secondary amplifier nucleic acids.

The population of secondary amplifier nucleic acids may be made using only 2 or only 3 of the 4 naturally occurring nucleotide bases, in certain embodiments such as leaving out all the “G”s or leaving out all of the “C”s within the population of nucleic acids. Sequences lacking either “G”s or “C”s may form very little secondary structure in certain embodiments, and can contribute to more uniform, faster hybridization. Thus, in some cases, the secondary amplifier nucleic acids may contain only A, T, and G; only A, T, and C; only A, C, and G; or only T, C, and G.

In some cases, more than one type of secondary amplifier nucleic acid may be applied to a sample, e.g., sequentially or simultaneously. For example, there may be at least 2, at least 5, at least 10, at least 25, at least 50, at least 75, at least 100, at least 300, at least 1,000, at least 3,000, at least 10,000, or at least 30,000 distinguishable secondary amplifier nucleic acids that are applied to a sample. In some cases, the secondary amplifier nucleic acids may be added sequentially. However, in some cases, more than one secondary amplifier nucleic acid may be added simultaneously.

In addition, in certain embodiments, this pattern can instead be repeated prior to the signaling entity, e.g., with tertiary amplifier nucleic acids, quaternary nucleic acids, etc., similar to the above discussion. The signaling entities may thus be bound to the ending amplifier nucleic acid. Thus, as non-limiting examples, to a target may be bound an encoding nucleic acid probe, to which a primary amplifier nucleic acid is bound, to which a secondary amplifier nucleic acid is bound, to which a tertiary amplifier nucleic acid is bound, to which a signaling entity is bound, or to a target may be bound an encoding nucleic acid probe, to which a primary amplifier nucleic acid is bound, to which a secondary amplifier nucleic acid is bound, to which a tertiary amplifier nucleic acid is bound, to which a quaternary amplifier nucleic acid is bound, to which a signaling entity is bound, etc. Accordingly, the ending amplifier nucleic acid need not necessarily be the secondary amplifier nucleic acid in all embodiments.

Other components may also be present within a nucleic acid probe or an amplifier nucleic acid as well. For example, in one set of embodiments, one or more primer sequences may be present, e.g., to facilitate enzymatic amplification. Those of ordinary skill in the art will be aware of primer sequences suitable for applications such as amplification (e.g., using PCR or other suitable techniques). Many such primer sequences are available commercially. Other examples of sequences that may be present within a primary nucleic acid probe include, but are not limited to promoter sequences, operons, identification sequences, nonsense sequences, or the like.

Typically, a primer is a single-stranded or partially double-stranded nucleic acid (e.g., DNA) that serves as a starting point for nucleic acid synthesis, allowing polymerase enzymes such as nucleic acid polymerase to extend the primer and replicate the complementary strand. A primer is (e.g., is designed to be) complementary to and to hybridize to a target nucleic acid. In some embodiments, a primer is a synthetic primer. In some embodiments, a primer is a non-naturally-occurring primer. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides.

In some embodiments, one or more signaling entities may be bound to the recognition entities on the secondary amplifier nucleic acids (or other ending amplifier nucleic acid). Non-limiting examples of signaling entities include fluorescent entities (fluorophores) or phosphorescent entities, e.g., as discussed below. The signaling entities may then be determined, e.g., to determine the nucleic acid probes or the targets. In some cases, the determination may be spatial, e.g., in two or three dimensions. In addition, in some cases, the determination may be quantitative, e.g., the amount or concentration of signaling entity and/or of a target may be determined.

In one set of embodiments, the signaling entities may be attached to the secondary amplifier nucleic acid (or other ending amplifier nucleic acid). The signaling entities may be attached to the secondary amplifier nucleic acid (or other ending amplifier nucleic acid) before or after association of the secondary amplifier nucleic acid to targets within the sample. For example, the signaling entities may be attached to the secondary amplifier nucleic acid initially, or after the secondary amplifier nucleic acids have been applied to a sample. In some cases, the signaling entities are added, then reacted to attach them to the amplifier nucleic acids.

In one set of embodiments, the signaling entities may be attached to a nucleotide sequence via a bond that can be cleaved to release the signaling entity. For example, after
determine the distribution of nucleic acid probes within a sample, the signaling entities may be released or inactivated, prior to another round of nucleic acid probes and/or amplifier nucleic acids. Thus, in some embodiments, the bond may be a cleavable bond, such as a disulfide bond or a photocleavable bond. Examples of photocleavable bonds are discussed in detail herein. In some cases, such bonds may be cleaved, for example, upon exposure to reducing agents or light (e.g., ultraviolet light). See below for additional details. Other examples of systems and methods for inactivating and/or removing the signaling entity are discussed in more detail herein.

In certain embodiments, the use of primary and secondary amplifier nucleic acids suggests that there is a maximum number of signaling entities that can be bound to a given nucleic acid probe. For instance, there may be a maximum number of primary amplifier nucleic acids is able to bind to a nucleic acid probe, e.g., due to a maximum number of secondary amplifier nucleic acids that are able to bind to a finite number of primary amplifier nucleic acids, and/or due to a maximum number of primary amplifier nucleic acids that are able to bind to the finite number of read sequences on the nucleic acid probes. While each potential location need not actually be filled with a signaling entity, this structure suggests that there is a saturation limit of signaling entities, beyond which any additional signaling entities that may happen to be present are unable to associate with a nucleic acid probe or its target.

Accordingly, certain embodiments of the disclosure are generally directed to systems and methods of amplifying a signal indicating a nucleic acid probe or its target that are saturable, i.e., such that there is an upper, saturation limit of how many signaling entities can associate with the nucleic acid probe or its target. Typically, that number is greater than 1. For instance, the upper limit of signaling entities may be at least 2, at least 3, at least 5, at least 10, at least 15, at least 20, at least 25, at least 30, at least 40, at least 50, at least 75, at least 100, at least 125, at least 150, at least 175, at least 200, at least 250, at least 300, at least 400, at least 500, etc. In some cases, the upper limit may be less than 500, less than 400, less than 300, less than 250, less than 200, less than 175, less than 150, less than 125, less than 100, less than 75, less than 50, less than 40, less than 30, less than 25, less than 20, less than 15, less than 10, less than 5, etc. In some cases, the upper limit may be determined as the maximum number of signaling entities that can bind to a secondary amplifier nucleic acid, multiplied by the maximum number of secondary amplifier nucleic acids that can bind to a primary amplifier nucleic acid, multiplied by the maximum number of primary amplifier nucleic acids that can bind to a nucleic acid probe that binds to a target. In contrast, techniques such as rolling circle amplification or hairpin unfolding allow the amplification of a signal in an uncontrolled manner, i.e., when sufficient reagents are present, amplification can continue without a predetermined endpoint or saturation limit. Thus, such techniques have no theoretical upper limit as to the number of signaling entities that can associate with the nucleic acid probe or its target.

It should be understood, however, that the average number of signaling entities actually bound to a nucleic acid probe or its target need not actually be the same as its upper limit, i.e., the signaling entities may not actually be at full saturation (although they can be). For instance, the amount of saturation (or the number of signaling entities bound, relative to the maximum number that can bind) may be less than 97%, less than 95%, less than 90%, less than 85%, less than 80%, less than 75%, etc., and/or at least 50%, at least 75%, at least 80%, at least 85%, at least 90%, at least 95%, at least 97%, etc. In some cases, allowing more time for binding to occur and/or increasing the concentration of reagents may increase the amount of saturation.

Because of the potential upper limit on the number of signaling entities actually bound to a nucleic acid probe or its target, the binding events distributed within a sample, e.g., spatially, may present substantially uniform sizes and/or brightnesses, in contrast to uncontrolled amplifications, such as those discussed above. For instance, due to the specific number of secondary amplifier nucleic acids that can bind to a primary amplifier nucleic acids, the secondary amplifier nucleic acids cannot be found greater than a fixed distance from the nucleic acid probe or its target, which may limit the “spot size” or diameter of fluorescence from the signaling entities, indicating binding.

In certain embodiments, at least 50%, at least 60%, at least 70%, at least 75%, at least 80%, at least 85%, at least 90%, or at least 95% of the binding events may exhibit substantially the same brightnesses, sizes (e.g., apparent diameters), colors, or the like, which may make it easier to distinguish binding events from other events, such as nonspecific binding, noise, or the like.

In addition, as previously discussed, certain aspects of the disclosure use code spaces that encode the various binding events, and optionally can use error detection and/or correction to determine the binding of nucleic acid probes to their targets. In some cases, a population of nucleic acid probes may contain certain “read sequences” which can bind certain amplifier nucleic acids, as discussed above, and the locations of the nucleic acid probes or targets can be determined within the sample using signaling entities associated with the amplifier nucleic acids, for example, within a certain code space, e.g., as discussed herein. See also Int. Pat. Apl. Pub. Nos. WO 2016/018960 and WO 2016/018963, each incorporated herein by reference in its entirety. As mentioned, in some cases, a population of read sequences within the nucleic acid probes may be combined in various combinations, e.g., such that a relatively small number of read sequences may be used to determine a relatively large number of different nucleic acid probes, as discussed herein.

Thus, in some cases, a population of nucleic acid probes may each contain a certain number of read sequences, some of which are shared between different nucleic acid probes such that the total population of nucleic acid probes may contain a certain number of read sequences. A population of nucleic acid probes may have any suitable number of read sequences. For example, a population of nucleic acid probes may have 1, 2, 3, 4, 5, 6, 7, 8, 9, 10, 11, 12, 13, 14, 15, 16, 17, 18, 19, 20, etc. read sequences. More than 20 are also possible in some embodiments. In addition, in some cases, a population of nucleic acid probes may, in total, have 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 20 or more, 24 or more, 32 or more, 40 or more, 50 or more, 60 or more, 64 or more, 100 or more, 128 or more, etc. of possible read sequences present, although some or all of the probes may each contain more than one read sequence, as discussed herein. In addition, in some embodiments, the population of nucleic acid probes may have no more than 100, no more than 80, no more than 64, no more than 60, no more than 50, no more than 40, no more than 32, no more than 24, no more than 20, no more than 16, no more than 15, no more than 14, no more than 13, no more than 12, no more than 11, no more than 10, no more than 9, no more than 8, no more than 7, no more than 6, no more than 5, no more than 4, no more than 3, or no more than two read sequences present. Combinations of any of these are also possible, e.g., a population of nucleic acid probes may comprise between 10 and 15 read sequences in total.

As a non-limiting example of an approach to combinatorially identifying a relatively large number of nucleic acid probes from a relatively small number of read sequences contained within the nucleic acid probes, in a population of 6 different types of nucleic acid probes, each comprising one or more read sequences, the total number of read sequences within the population may be no greater than 4. It should be understood that although 4 read sequences are used in this example for ease of explanation, in other embodiments, larger numbers of nucleic acid probes may be realized, for example, using 5, 8, 10, 16, 32, etc. or more read sequences, or any other suitable number of read sequences described herein, depending on the application. For example, if each of the nucleic acid probes contains two different read sequences, then by using 4 such read sequences (A, B, C, and D), up to 6 probes may be separately identified. It should be noted that in this example, the ordering of read sequences on a nucleic acid probe is not essential, i.e., “AB” and “BA” may be treated as being synonymous (although in other embodiments, the ordering of read sequences may be essential and “AB” and “BA” may not necessarily be synonymous). Similarly, if 5 read sequences are used (A, B, C, D, and E) in the population of nucleic acid probes, up to 10 probes may be separately identified (e.g., AB, AC, AD, AE, BC, BD, BE, CD, CE, DE). For example, one of ordinary skill in the art would understand that, for k read sequences in a population with n read sequences on each probe, up to

$$( \begin{matrix}n \\ k\end{matrix} )$$

different probes may be produced, assuming that the ordering of read sequences is not essential; because not all of the probes need to have the same number of read sequences and not all combinations of read sequences need to be used in every embodiment, either more or less than this number of different probes may also be used in certain embodiments. In addition, it should also be understood that the number of read sequences on each probe need not be identical in some embodiments. For instance example, some probes may contain 2 read sequences while other probes may contain 3 read sequences.

In some aspects, the read sequences and/or the pattern of binding of nucleic acid probes within a sample may be used to define an error-detecting and/or an error-correcting code, for example, to reduce or prevent misidentification or errors of the nucleic acids. As would be known by those of ordinary skill in the art, an “error-detecting code” is a code that allows for the detection of errors caused by noise or other impairments during transmission, while an “error-correction code” is similar to an error-detecting code, but the code further allows for the reconstruction of the original data. Thus, for example, if binding is indicated (e.g., as determined using a signaling entity), then the location may be identified with a “1”; conversely, if no binding is indicated, then the location may be identified with a “0” (or vice versa, in some cases). Multiple rounds of binding determinations, e.g., using different nucleic acid probes, can then be used to create a “codeword,” e.g., for that spatial location. The “codeword” is a numerical string of digits, where each digit represents a location representing a binding determination. For example, as discussed herein, 3 rounds of binding determinations may be used to create codewords that are 3 digits long, each digit representing one round of binding. Codewords with other lengths are also possible in other embodiments. The space of all possible values for the codewords may define a code space.

In some embodiments, the codeword may be subjected to error detection and/or correction. For instance, the codewords may be organized such that, if no match is found for a given set of read sequences or binding pattern of nucleic acid probes, then the match may be identified as an error, and optionally, error correction may be applied sequences to determine the correct target for the nucleic acid probes. In some cases, the codewords may have fewer “letters” or positions that the total number of nucleic acids encoded by the codewords, e.g. where each codeword encodes a different nucleic acid.

Such error-detecting and/or the error-correction code may take a variety of forms. A variety of such codes have previously been developed in other contexts such as the telecommunications industry, such as Golay codes or Hamming codes. In one set of embodiments, the read sequences or binding patterns of the nucleic acid probes are assigned such that not every possible combination is assigned.

For example, if 4 read sequences are possible and a nucleic acid probe contains 2 read sequences, then up to 6 nucleic acid probes could be identified; but the number of nucleic acid probes used may be less than 6. Similarly, for k read sequences in a population with n read sequences on each nucleic acid probe,

$$( \begin{matrix}n \\ k\end{matrix} )$$

different probes may be produced, but the number of nucleic acid probes that are used may be any number more or less than

$$( \begin{matrix}n \\ k\end{matrix} ) .$$

In addition, these may be randomly assigned, or assigned in specific ways to increase the ability to detect and/or correct errors.

As another example, if multiple rounds of nucleic acid probes are used, the number of rounds may be arbitrarily chosen. If in each round, each target can give two possible outcomes, such as being detected or not being detected, up to 2n different targets may be possible for n rounds of probes, but the number of targets that are actually used may be any number less than 2n. For example, if in each round, each target can give more than two possible outcomes, such as being detected in different color channels, more than 2n (e.g. 3n, 4n, . . . ) different targets may be possible for n rounds of probes. In some cases, the number of targets that are actually used may be any number less than this number. In addition, these may be randomly assigned, or assigned in specific ways to increase the ability to detect and/or correct errors.

The codewords may be used to define various code spaces. For example, in one set of embodiments, the codewords or nucleic acid probes may be assigned within a code space such that the assignments are separated by a Hamming distance, which measures the number of incorrect “reads” in a given pattern that cause the nucleic acid probe to be misinterpreted as a different valid nucleic acid probe. In certain cases, the Hamming distance may be at least 2, at least 3, at least 4, at least 5, at least 6, or the like. In addition, in one set of embodiments, the assignments may be formed as a Hamming code, for instance, a Hamming (7, 4) code, a Hamming (15, 11) code, a Hamming (31, 26) code, a Hamming (63, 57) code, a Hamming (127, 120) code, etc. In another set of embodiments, the assignments may form a SECDED code, e.g., a SECDED (8,4) code, a SECDED (16,4) code, a SCEDED (16, 11) code, a SCEDED (22, 16) code, a SCEDED (39, 32) code, a SCEDED (72, 64) code, etc. In yet another set of embodiments, the assignments may form an extended binary Golay code, a perfect binary Golay code, or a ternary Golay code. In another set of embodiments, the assignments may represent a subset of the possible values taken from any of the codes described above.

For example, an error-correcting code may be formed by using only binary words that contain a fixed or constant number of “1” bits (or “0” bits) to encode the targets. For example, the code space may only include 1, 2, 3, 4, 5, 6, 7, 8, 9, 10, 11, 12, 13, 14, 15, 16, etc. “1” bits (or “0” bits), e.g., all of the codes have the same number of “1” bits or “0” bits, etc. In another set of embodiments, the assignments may represent a subset of the possible values taken from codes described above for the purpose of addressing asymmetric readout errors. For example, in some cases, a code in which the number of “1” bits may be fixed for all used binary words may eliminate the biased measurement of words with different numbers of “1”s when the rate at which “0” bits are measured as “1”s or “1” bits are measured as “0”s are different.

Accordingly, in some embodiments, once the codeword is determined (e.g., as discussed herein), the codeword may be compared to the known nucleic acid codewords. If a match is found, then the nucleic acid target can be identified or determined. If no match is found, then an error in the reading of the codeword may be identified. In some cases, error correction can also be applied to determine the correct codeword, and thus resulting in the correct identity of the nucleic acid target. In some cases, the codewords may be selected such that, assuming that there is only one error present, only one possible correct codeword is available, and thus, only one correct identity of the nucleic acid target is possible. In some cases, this may also be generalized to larger codeword spacings or Hamming distances; for instance, the codewords may be selected such that if two, three, or four errors are present (or more in some cases), only one possible correct codeword is available, and thus, only one correct identity of the nucleic acid targets is possible.

The error-correcting code may be a binary error-correcting code, or it may be based on other numbering systems, e.g., ternary or quaternary error-correcting codes. For instance, in one set of embodiments, more than one type of signaling entity may be used and assigned to different numbers within the error-correcting code. Thus, as a non-limiting example, a first signaling entity (or more than one signaling entity, in some cases) may be assigned as “1” and a second signaling entity (or more than one signaling entity, in some cases) may be assigned as “2” (with “0” indicating no signaling entity present), and the codewords distributed to define a ternary error-correcting code. Similarly, a third signaling entity may additionally be assigned as “3” to make a quaternary error-correcting code, etc.

As discussed herein, in certain aspects, signaling entities are determined, e.g., by imaging, to determine nucleic acid probes and/or to create codewords. Examples of signaling entities include those discussed herein. In some cases, signaling entities within a sample may be determined, e.g., spatially, using a variety of techniques. In some embodiments, the signaling entities may be fluorescent, and techniques for determining fluorescence within a sample, such as fluorescence microscopy or confocal microscopy, may be used to spatially identify the positions of signaling entities within a cell. In some cases, the positions of entities within the sample may be determined in two or even three dimensions. In addition, in some embodiments, more than one signaling entity may be determined at a time (e.g., signaling entities with different colors or emissions), and/or sequentially.

In addition, in some embodiments, a confidence level for an identified target, e.g., a nucleic acid target, may be determined. For example, the confidence level may be determined using a ratio of the number of exact matches to the number of matches having one or more one-bit errors. In some cases, only matches having a confidence ratio greater than a certain value may be used. For instance, in certain embodiments, matches may be accepted only if the confidence ratio for the match is greater than about 0.01, greater than about 0.03, greater than about 0.05, greater than about 0.1, greater than about 0.3, greater than about 0.5, greater than about 1, greater than about 3, greater than about 5, greater than about 10, greater than about 30, greater than about 50, greater than about 100, greater than about 300, greater than about 500, greater than about 1000, or any other suitable value. In addition, in some embodiments, matches may be accepted only if the confidence ratio for the identified target is greater than an internal standard or false positive control by about 0.01, about 0.03, about 0.05, about 0.1, about 0.3, about 0.5, about 1, about 3, about 5, about 10, about 30, about 50, about 100, about 300, about 500, about 1000, or any other suitable value

In some embodiments, the spatial positions of the entities (and thus, nucleic acid probes that the entities may be associated with) may be determined at relatively high resolutions. For instance, the positions may be determined at spatial resolutions of better than about 100 micrometers, better than about 30 micrometers, better than about 10 micrometers, better than about 3 micrometers, better than about 1 micrometer, better than about 800 nm, better than about 600 nm, better than about 500 nm, better than about 400 nm, better than about 300 nm, better than about 200 nm, better than about 100 nm, better than about 90 nm, better than about 80 nm, better than about 70 nm, better than about 60 nm, better than about 50 nm, better than about 40 nm, better than about 30 nm, better than about 20 nm, or better than about 10 nm, etc.

There are a variety of techniques able to determine or image the spatial positions of entities optically, e.g., using fluorescence microscopy. More than one color can be used in some embodiments. In some cases, the spatial positions may be determined at super resolutions, or at resolutions better than the wavelength of light or the diffraction limit. Non-limiting examples include STORM (stochastic optical reconstruction microscopy), STED (stimulated emission depletion microscopy), NSOM (Near-field Scanning Optical Microscopy), 4Pi microscopy, SIM (Structured Illumination Microscopy), SMI (Spatially Modulated Illumination) microscopy, RESOLFT (Reversible Saturable Optically Linear Fluorescence Transition Microscopy), GSD (Ground State Depletion Microscopy), SSIM (Saturated Structured-Illumination Microscopy), SPDM (Spectral Precision Distance Microscopy), Photo-Activated Localization Microscopy (PALM), Fluorescence Photoactivation Localization Microscopy (FPALM), LIMON (3D Light Microscopical Nanosizing Microscopy), Super-resolution optical fluctuation imaging (SOFI), or the like. See, e.g., U.S. Pat. No. 7,838,302, issued Nov. 23, 2010, entitled “Sub-Diffraction Limit Image Resolution and Other Imaging Techniques,” by Zhuang, et al.; U.S. Pat. No. 8,564,792, issued Oct. 22, 2013, entitled “Sub-diffraction Limit Image Resolution in Three Dimensions,” by Zhuang, et al.; or Int. Pat. Apl. Pub. No. WO 2013/090360, published Jun. 20, 2013, entitled “High Resolution Dual-Objective Microscopy,” by Zhuang, et al., each incorporated herein by reference in their entireties.

As an illustrative non-limiting example, in one set of embodiments, the sample may be imaged with a high numerical aperture, oil immersion objective with 100× magnification and light collected on an electron-multiplying CCD camera. In another example, the sample could be imaged with a high numerical aperture, oil immersion lens with 40× magnification and light collected with a wide-field scientific CMOS camera. With different combinations of objectives and cameras, a single field of view may correspond to no less than 40×40 microns, 80×80 microns, 120×120 microns, 240×240 microns, 340×340 microns, or 500×500 microns, etc. in various non-limiting embodiments. Similarly, a single camera pixel may correspond, in some embodiments, to regions of the sample of no less than 80×80 nm, 120×120 nm, 160×160 nm, 240×240 nm, or 300×300 nm, etc. In another example, the sample may be imaged with a low numerical aperture, air lens with 10× magnification and light collected with a sCMOS camera. In additional embodiments, the sample may be optically sectioned by illuminating it via a single or multiple scanned diffraction limited foci generated either by scanning mirrors or a spinning disk and the collected passed through a single or multiple pinholes. In another embodiment, the sample may also be illuminated via thin sheet of light generated via any one of multiple methods known to those versed in the art.

In one embodiment, the sample may be illuminated by single Gaussian mode laser lines. In some embodiments, the illumination profiled may be flattened by passing these laser lines through a multimode fiber that is vibrated via piezo-electric or other mechanical means. In some embodiments, the illumination profile may be flattened by passing single-mode, Gaussian beams through a variety of refractive beam shapers, such as the piShaper or a series of stacked Powell lenses. In yet another set of embodiments, the Gaussian beams may be passed through a variety of different diffusing elements, such as ground glass or engineered diffusers, which may be spun in some cases at high speeds to remove residual laser speckle. In yet another embodiment, laser illumination may be passed through a series of lenslet arrays to produce overlapping images of the illumination that approximate a flat illumination field.

In some embodiments, the centroids of the spatial positions of the entities may be determined. For example, a centroid of a signaling entity may be determined within an image or series of images using image analysis algorithms known to those of ordinary skill in the art. In some cases, the algorithms may be selected to determine non-overlapping single emitters and/or partially overlapping single emitters in a sample. Non-limiting examples of suitable techniques include a maximum likelihood algorithm, a least squares algorithm, a Bayesian algorithm, a compressed sensing algorithm, or the like. Combinations of these techniques may also be used in some cases.

In addition, the signaling entity may be inactivated in some cases. For example, in some embodiments, a first secondary nucleic acid probe that can associate with a signaling entity (e.g., using amplifier nucleic acids) may be applied to a sample that can recognize a first read sequence (e.g., on the nucleic acid probe), then the signaling entity can be inactivated before a second secondary nucleic acid probe is applied to the sample, e.g., that can associate with a signaling entity (e.g., using amplifier nucleic acids). If multiple signaling entities are used, the same or different techniques may be used to inactivate the signaling entities, and some or all of the multiple signaling entities may be inactivated, e.g., sequentially or simultaneously.

Inactivation may be caused by removal of the signaling entity (e.g., from the sample, or from the nucleic acid probe, etc.), and/or by chemically altering the signaling entity in some fashion (e.g., by photobleaching the signaling entity, bleaching or chemically altering the structure of the signaling entity, for example, by reduction, etc.). For instance, in one set of embodiments, a fluorescent signaling entity may be inactivated by chemical or optical techniques such as oxidation, photobleaching, chemically bleaching, stringent washing or enzymatic digestion or reaction by exposure to an enzyme, dissociating the signaling entity from other components (e.g., a probe), chemical reaction of the signaling entity (e.g., to a reactant able to alter the structure of the signaling entity) or the like. For instance, bleaching may occur by exposure to oxygen, reducing agents, or the signaling entity could be chemically cleaved from the nucleic acid probe and washed away via fluid flow.

In some embodiments, various nucleic acid probes may be associated with one or more signaling entities, e.g., using amplifier nucleic acids as discussed herein. If more than one nucleic acid probe is used, the signaling entities may each by the same or different. In certain embodiments, a signaling entity is any entity able to emit light. For instance, in one embodiment, the signaling entity is fluorescent. In other embodiments, the signaling entity may be phosphorescent, radioactive, absorptive, etc. In some cases, the signaling entity is any entity that can be determined within a sample at relatively high resolutions, e.g., at resolutions better than the wavelength of visible light or the diffraction limit. The signaling entity may be, for example, a dye, a small molecule, a peptide or protein, or the like. The signaling entity may be a single molecule in some cases. If multiple secondary nucleic acid probes are used, the nucleic acid probes may associate with the same or different signaling entities.

Non-limiting examples of signaling entities include fluorescent entities (fluorophores) or phosphorescent entities, for example, cyanine dyes (e.g., Cy2, Cy3, Cy3B, Cy5, Cy5.5, Cy7, etc.), Alexa Fluor dyes, Atto dyes, photoswitchable dyes, photoactivatable dyes, fluorescent dyes, metal nanoparticles, semiconductor nanoparticles or “quantum dots,” fluorescent proteins such as GFP (Green Fluorescent Protein), or photoactivabale fluorescent proteins, such as PAGFP, PSCFP, PSCFP2, Dendra, Dendra2, EosFP, tdEos, mEos2, mEos3, PAmCherry, PAtagRFP, mMaple, mMaple2, and mMaple3. Other suitable signaling entities are known to those of ordinary skill in the art. See, e.g., U.S. Pat. No. 7,838,302 or Int. Pat Apl. Pub. No. WO 2015/160690, each incorporated herein by reference in its entirety.

In one set of embodiments, the signaling entity may be attached to an oligonucleotide sequence via a bond that can be cleaved to release the signaling entity. In one set of embodiments, a fluorophore may be conjugated to an oligonucleotide via a cleavable bond, such as a photocleavable bond. Non-limiting examples of photocleavable bonds include, but are not limited to, 1-(2-nitrophenyl)ethyl, 2-nitrobenzyl, biotin phosphoramidite, acrylic phosphoramidite, diethylaminocoumarin, 1-(4,5-dimethoxy-2-nitrophenyl) ethyl, cyclo-dodecyl (dimethoxy-2-nitrophenyl)ethyl, 4-aminomethyl-3-nitrobenzyl, (4-nitro-3-(1-chlorocarbonyloxyethyl)phenyl)methyl-S-acetylthioic acid ester, (4-nitro-3-(1-thlorocarbonyloxyethyl)phenyl)methyl-3-(2-pyridyldithiopropionic acid) ester, 3-(4,4′-dimethoxytrityl)-1-(2-nitrophenyl)-propane-1,3-diol-[2-cyanoethyl-(N,N-diisopropyl)]-phosphoramidite, 1-[2-nitro-5-(6-trifluoroacetylcaproamidomethyl)phenyl]-ethyl-[2-cyano-ethyl-(N,N-diisopropyl)]-phosphoramidite, 1-[2-nitro-5-(6-(4,4′-dimethoxytrityloxy)butyramidomethyl)phenyl]-ethyl-[2-cyanoethyl-(N,N-diisopropyl)]-phosphoramidite, 1-[2-nitro-5-(6-(N-(4,4′-dimethoxytrityl))-biotinamidocaproamido-methyl)phenyl]-ethyl-[2-cyanoethyl-(N,N-diisopropyl)]-phosphoramidite, or similar linkers. The oligonucleotide sequence may be, for example, a primary or secondary (or other) amplifier nucleic acid, such as those discussed herein.

In another set of embodiments, the fluorophore may be conjugated to an oligonucleotide via a disulfide bond. The disulfide bond may be cleaved by a variety of reducing agents such as, but not limited to, dithiothreitol, dithioerythritol, beta-mercaptoethanol, sodium borohydride, thioredoxin, glutaredoxin, trypsinogen, hydrazine, diisobutylaluminum hydride, oxalic acid, formic acid, ascorbic acid, phosphorous acid, tin chloride, glutathione, thioglycolate, 2,3-dimercaptopropanol, 2-mercaptoethylamine, 2-aminoethanol, tris(2-carboxyethyl)phosphine, bis(2-mercaptoethyl) sulfone, N,N′-dimethyl-N,N′-bis(mercaptoacetyl)hydrazine, 3-mercaptoproptionate, dimethylformamide, thiopropyl-agarose, tri-n-butylphosphine, cysteine, iron sulfate, sodium sulfite, phosphite, hypophosphite, phosphorothioate, or the like, and/or combinations of any of these. The oligonucleotide sequence may be, for example, a primary or secondary (or other) amplifier nucleic acid, such as those discussed herein.

In another embodiment, the fluorophore may be conjugated to an oligonucleotide via one or more phosphorothioate modified nucleotides in which the sulfur modification replaces the bridging and/or non-bridging oxygen. The fluorophore may be cleaved from the oligonucleotide, in certain embodiments, via addition of compounds such as but not limited to iodoethanol, iodine mixed in ethanol, silver nitrate, or mercury chloride. In yet another set of embodiments, the signaling entity may be chemically inactivated through reduction or oxidation. For example, in one embodiment, a chromophore such as Cy5 or Cy7 may be reduced using sodium borohydride to a stable, non-fluorescence state. In still another set of embodiments, a fluorophore may be conjugated to an oligonucleotide via an azo bond, and the azo bond may be cleaved with 2-[(2-N-arylamino)phenylazo]pyridine. In yet another set of embodiments, a fluorophore may be conjugated to an oligonucleotide via a suitable nucleic acid segment that can be cleaved upon suitable exposure to DNAse, e.g., an exodeoxyribonuclease or an endodeoxyribonuclease. Examples include, but are not limited to, deoxyribonuclease I or deoxyribonuclease II. In one set of embodiments, the cleavage may occur via a restriction endonuclease. Non-limiting examples of potentially suitable restriction endonucleases include BamHI, BsrI, NotI, XmaI, PspAI, DpnI, MboI, MnlI, Eco57I, Ksp632I, DraIII, AhaII, SmaI, MluI, HpaI, ApaI, BclI, BstEII, TaqI, EcoRI, SacI, HindII, HaeII, DraII, Tsp509I, Sau3AI, PacI, etc. Over 3000 restriction enzymes have been studied in detail, and more than 600 of these are available commercially. In yet another set of embodiments, a fluorophore may be conjugated to biotin, and the oligonucleotide conjugated to avidin or streptavidin. An interaction between biotin and avidin or streptavidin allows the fluorophore to be conjugated to the oligonucleotide, while sufficient exposure to an excess of addition, free biotin could “outcompete” the linkage and thereby cause cleavage to occur. In addition, in another set of embodiments, the probes may be removed using corresponding “toe-hold-probes,” which comprise the same sequence as the probe, as well as an extra number of bases of homology to the encoding probes (e.g., 1-20 extra bases, for example, 5 extra bases). These probes may remove the labeled readout probe through a strand-displacement interaction. The oligonucleotide sequence may be, for example, a primary or secondary (or other) amplifier nucleic acid, such as those discussed herein.

As used herein, the term “light” generally refers to electromagnetic radiation, having any suitable wavelength (or equivalently, frequency). For instance, in some embodiments, the light may include wavelengths in the optical or visual range (for example, having a wavelength of between about 400 nm and about 700 nm, i.e., “visible light”), infrared wavelengths (for example, having a wavelength of between about 300 micrometers and 700 nm), ultraviolet wavelengths (for example, having a wavelength of between about 400 nm and about 10 nm), or the like. In certain cases, as discussed in detail below, more than one entity may be used, i.e., entities that are chemically different or distinct, for example, structurally. However, in other cases, the entities may be chemically identical or at least substantially chemically identical.

In one set of embodiments, the signaling entity is “switchable,” i.e., the entity can be switched between two or more states, at least one of which emits light having a desired wavelength. In the other state(s), the entity may emit no light, or emit light at a different wavelength. For instance, an entity may be “activated” to a first state able to produce light having a desired wavelength, and “deactivated” to a second state not able to emit light of the same wavelength. An entity is “photoactivatable” if it can be activated by incident light of a suitable wavelength. As a non-limiting example, Cy5, can be switched between a fluorescent and a dark state in a controlled and reversible manner by light of different wavelengths, i.e., 633 nm (or 642 nm, 647 nm, 656 nm) red light can switch or deactivate Cy5 to a stable dark state, while 405 nm green light can switch or activate the Cy5 back to the fluorescent state. In some cases, the entity can be reversibly switched between the two or more states, e.g., upon exposure to the proper stimuli. For example, a first stimuli (e.g., a first wavelength of light) may be used to activate the switchable entity, while a second stimuli (e.g., a second wavelength of light) may be used to deactivate the switchable entity, for instance, to a non-emitting state. Any suitable method may be used to activate the entity. For example, in one embodiment, incident light of a suitable wavelength may be used to activate the entity to emit light, i.e., the entity is “photoswitchable.” Thus, the photoswitchable entity can be switched between different light-emitting or non-emitting states by incident light, e.g., of different wavelengths. The light may be monochromatic (e.g., produced using a laser) or polychromatic. In another embodiment, the entity may be activated upon stimulation by electric field and/or magnetic field. In other embodiments, the entity may be activated upon exposure to a suitable chemical environment, e.g., by adjusting the pH, or inducing a reversible chemical reaction involving the entity, etc. Similarly, any suitable method may be used to deactivate the entity, and the methods of activating and deactivating the entity need not be the same. For instance, the entity may be deactivated upon exposure to incident light of a suitable wavelength, or the entity may be deactivated by waiting a sufficient time.

Typically, a “switchable” entity can be identified by one of ordinary skill in the art by determining conditions under which an entity in a first state can emit light when exposed to an excitation wavelength, switching the entity from the first state to the second state, e.g., upon exposure to light of a switching wavelength, then showing that the entity, while in the second state can no longer emit light (or emits light at a much reduced intensity) when exposed to the excitation wavelength.

In one set of embodiments, as discussed, a switchable entity may be switched upon exposure to light. In some cases, the light used to activate the switchable entity may come from an external source, e.g., a light source such as a laser light source, another light-emitting entity proximate the switchable entity, etc. The second, light emitting entity, in some cases, may be a fluorescent entity, and in certain embodiments, the second, light-emitting entity may itself also be a switchable entity.

In some embodiments, the switchable entity includes a first, light-emitting portion (e.g., a fluorophore), and a second portion that activates or “switches” the first portion. For example, upon exposure to light, the second portion of the switchable entity may activate the first portion, causing the first portion to emit light. Examples of activator portions include, but are not limited to, Alexa Fluor 405 (Invitrogen), Alexa Fluor 488 (Invitrogen), Cy2 (GE Healthcare), Cy3 (GE Healthcare), Cy3B (GE Healthcare), Cy3.5 (GE Healthcare), or other suitable dyes. Examples of light-emitting portions include, but are not limited to, Cy5, Cy5.5 (GE Healthcare), Cy7 (GE Healthcare), Alexa Fluor 647 (Invitrogen), Alexa Fluor 680 (Invitrogen), Alexa Fluor 700 (Invitrogen), Alexa Fluor 750 (Invitrogen), Alexa Fluor 790 (Invitrogen), DiD, DiR, YOYO-3 (Invitrogen), YO-PRO-3 (Invitrogen), TOT-3 (Invitrogen), TO-PRO-3 (Invitrogen) or other suitable dyes. These may linked together, e.g., covalently, for example, directly, or through a linker, e.g., forming compounds such as, but not limited to, Cy5-Alexa Fluor 405, Cy5-Alexa Fluor 488, Cy5-Cy2, Cy5-Cy3, Cy5-Cy3.5, Cy5.5-Alexa Fluor 405, Cy5.5-Alexa Fluor 488, Cy5.5-Cy2, Cy5.5-Cy3, Cy5.5-Cy3.5, Cy7-Alexa Fluor 405, Cy7-Alexa Fluor 488, Cy7-Cy2, Cy7-Cy3, Cy7-Cy3.5, Alexa Fluor 647-Alexa Fluor 405, Alexa Fluor 647-Alexa Fluor 488, Alexa Fluor 647-Cy2, Alexa Fluor 647-Cy3, Alexa Fluor 647-Cy3.5, Alexa Fluor 750-Alexa Fluor 405, Alexa Fluor 750-Alexa Fluor 488, Alexa Fluor 750-Cy2, Alexa Fluor 750-Cy3, or Alexa Fluor 750-Cy3.5. Those of ordinary skill in the art will be aware of the structures of these and other compounds, many of which are available commercially. The portions may be linked via a covalent bond, or by a linker, such as those described in detail below. Other light-emitting or activator portions may include portions having two quaternized nitrogen atoms joined by a polymethine chain, where each nitrogen is independently part of a heteroaromatic moiety, such as pyrrole, imidazole, thiazole, pyridine, quinoine, indole, benzothiazole, etc., or part of a nonaromatic amine. In some cases, there may be 5, 6, 7, 8, 9, or more carbon atoms between the two nitrogen atoms.

In certain cases, the light-emitting portion and the activator portions, when isolated from each other, may each be fluorophores, i.e., entities that can emit light of a certain, emission wavelength when exposed to a stimulus, for example, an excitation wavelength. However, when a switchable entity is formed that comprises the first fluorophore and the second fluorophore, the first fluorophore forms a first, light-emitting portion and the second fluorophore forms an activator portion that switches that activates or “switches” the first portion in response to a stimulus. For example, the switchable entity may comprise a first fluorophore directly bonded to the second fluorophore, or the first and second entity may be connected via a linker or a common entity. Whether a pair of light-emitting portion and activator portion produces a suitable switchable entity can be tested by methods known to those of ordinary skills in the art. For example, light of various wavelength can be used to stimulate the pair and emission light from the light-emitting portion can be measured to determined wither the pair makes a suitable switch.

As a non-limiting example, Cy3 and Cy5 may be linked together to form such an entity. In this example, Cy3 is an activator portion that is able to activate Cy5, the light-emission portion. Thus, light at or near the absorption maximum (e.g., near 532 nm light for Cy3) of the activation or second portion of the entity may cause that portion to activate the first, light-emitting portion, thereby causing the first portion to emit light (e.g., near 647 nm for Cy5). See, e.g., U.S. Pat. No. 7,838,302, incorporated herein by reference in its entirety. In some cases, the first, light-emitting portion can subsequently be deactivated by any suitable technique (e.g., by directing 647 nm red light to the Cy5 portion of the molecule).

Other non-limiting examples of potentially suitable activator portions include 1,5 IAEDANS, 1,8-ANS, 4-Methylumbelliferone, 5-carboxy-2,7-dichlorofluorescein, 5-Carboxyfluorescein (5-FAM), 5-Carboxynapthofluorescein, 5-Carboxytetramethylrhodamine (5-TAMRA), 5-FAM (5-Carboxyfluorescein), 5-HAT (Hydroxy Tryptamine), 5-Hydroxy Tryptamine (HAT), 5-ROX (carboxy-X-rhodamine), 5-TAMRA (5-Carboxytetramethylrhodamine), 6-Carboxyrhodamine 6G, 6-CR 6G, 6-JOE, 7-Amino-4-methylcoumarin, 7-Aminoactinomycin D (7-AAD), 7-Hydroxy-4-methylcoumarin, 9-Amino-6-chloro-2-methoxyacridine, ABQ, Acid Fuchsin, ACMA (9-Amino-6-chloro-2-methoxyacridine), Acridine Orange, Acridine Red, Acridine Yellow, Acriflavin, Acriflavin Feulgen SITSA, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alizarin Complexon, Alizarin Red, AMC, AMCA-S, AMCA (Aminomethylcoumarin), AMCA-X, Aminoactinomycin D, Aminocoumarin, Aminomethylcoumarin (AMCA), Anilin Blue, Anthrocyl stearate, APTRA-BTC, APTS, Astrazon Brilliant Red 4G, Astrazon Orange R, Astrazon Red 6B, Astrazon Yellow 7 GLL, Atabrine, ATTO 390, ATTO 425, ATTO 465, ATTO 488, ATTO 495, ATTO 520, ATTO 532, ATTO 550, ATTO 565, ATTO 590, ATTO 594, ATTO 610, ATTO 611X, ATTO 620, ATTO 633, ATTO 635, ATTO 647, ATTO 647N, ATTO 655, ATTO 680, ATTO 700, ATTO 725, ATTO 740, ATTO-TAG CBQCA, ATTO-TAG FQ, Auramine, Aurophosphine G, Aurophosphine, BAO 9 (Bisaminophenyloxadiazole), BCECF (high pH), BCECF (low pH), Berberine Sulphate, Bimane, Bisbenzamide, Bisbenzimide (Hoechst), bis-BTC, Blancophor FFG, Blancophor SV, BOBO-1, BOBO-3, Bodipy 492/515, Bodipy 493/503, Bodipy 500/510, Bodipy 505/515, Bodipy 530/550, Bodipy 542/563, Bodipy 558/568, Bodipy 564/570, Bodipy 576/589, Bodipy 581/591, Bodipy 630/650-X, Bodipy 650/665-X, Bodipy 665/676, Bodipy Fl, Bodipy FL ATP, Bodipy Fl-Ceramide, Bodipy R6G, Bodipy TMR, Bodipy TMR-X conjugate, Bodipy TMR-X, SE, Bodipy TR, Bodipy TR ATP, Bodipy TR-X SE, BO-PRO-1, BO-PRO-3, Brilliant Sulphoflavin FF, BTC, BTC-5N, Calcein, Calcein Blue, Calcium Crimson, Calcium Green, Calcium Green-1 Ca2+ Dye, Calcium Green-2 Ca2+, Calcium Green-5N Ca2+, Calcium Green-C18 Ca2+, Calcium Orange, Calcofluor White, Carboxy-X-rhodamine (5-ROX), Cascade Blue, Cascade Yellow, Catecholamine, CCF2 (GeneBlazer), CFDA, Chromomycin A, Chromomycin A, CL-NERF, CMFDA, Coumarin Phalloidin, CPM Methylcoumarin, CTC, CTC Formazan, Cy2, Cy3.1 8, Cy3.5, Cy3, Cy5.1 8, cyclic AMP Fluorosensor (FiCRhR), Dabcyl, Dansyl, Dansyl Amine, Dansyl Cadaverine, Dansyl Chloride, Dansyl DHPE, Dansyl fluoride, DAPI, Dapoxyl, Dapoxyl 2, Dapoxyl 3′ DCFDA, DCFH (Dichlorodihydrofluorescein Diacetate), DDAO, DHR (Dihydorhodamine 123), Di-4-ANEPPS, Di-8-ANEPPS (non-ratio), DiA (4-Di-16-ASP), Dichlorodihydrofluorescein Diacetate (DCFH), DiD—Lipophilic Tracer, DiD (DiIC18(5)), DIDS, Dihydorhodamine 123 (DHR), DiI (DiIC18(3)), Dinitrophenol, DiO (DiOC18(3)), DiR, DiR (DiIC18(7)), DM-NERF (high pH), DNP, Dopamine, DTAF, DY-630-NHS, DY-635-NHS, DyLight 405, DyLight 488, DyLight 549, DyLight 633, DyLight 649, DyLight 680, DyLight 800, ELF 97, Eosin, Erythrosin, Erythrosin ITC, Ethidium Bromide, Ethidium homodimer-1 (EthD-1), Euchrysin, EukoLight, Europium (III) chloride, Fast Blue, FDA, Feulgen (Pararosaniline), FIF (Formaldehyd Induced Fluorescence), FITC, Flazo Orange, Fluo-3, Fluo-4, Fluorescein (FITC), Fluorescein Diacetate, Fluoro-Emerald, Fluoro-Gold (Hydroxystilbamidine), Fluor-Ruby, FluorX, FM 1-43, FM 4-46, Fura Red (high pH), Fura Red/Fluo-3, Fura-2, Fura-2/BCECF, Genacryl Brilliant Red B, Genacryl Brilliant Yellow 10GF, Genacryl Pink 3G, Genacryl Yellow 5GF, GeneBlazer (CCF2), Gloxalic Acid, Granular blue, Haematoporphyrin, Hoechst 33258, Hoechst 33342, Hoechst 34580, HPTS, Hydroxycoumarin, Hydroxystilbamidine (FluoroGold), Hydroxytryptamine, Indo-1, high calcium, Indo-1, low calcium, Indodicarbocyanine (DiD), Indotricarbocyanine (DiR), Intrawhite Cf, JC-1, JO-JO-1, JO-PRO-1, LaserPro, Laurodan, LDS 751 (DNA), LDS 751 (RNA), Leucophor PAF, Leucophor SF, Leucophor WS, Lissamine Rhodamine, Lissamine Rhodamine B, Calcein/Ethidium homodimer, LOLO-1, LO-PRO-1, Lucifer Yellow, Lyso Tracker Blue, Lyso Tracker Blue-White, Lyso Tracker Green, Lyso Tracker Red, Lyso Tracker Yellow, LysoSensor Blue, LysoSensor Green, LysoSensor Yellow/Blue, Mag Green, Magdala Red (Phloxin B), Mag-Fura Red, Mag-Fura-2, Mag-Fura-5, Mag-Indo-1, Magnesium Green, Magnesium Orange, Malachite Green, Marina Blue, Maxilon Brilliant Flavin 10 GFF, Maxilon Brilliant Flavin 8 GFF, Merocyanin, Methoxycoumarin, Mitotracker Green FM, Mitotracker Orange, Mitotracker Red, Mitramycin, Monobromobimane, Monobromobimane (mBBr-GSH), Monochlorobimane, MPS (Methyl Green Pyronine Stilbene), NBD, NBD Amine, Nile Red, Nitrobenzoxadidole, Noradrenaline, Nuclear Fast Red, Nuclear Yellow, Nylosan Brilliant lavin E8G, Oregon Green, Oregon Green 488-X, Oregon Green, Oregon Green 488, Oregon Green 500, Oregon Green 514, Pacific Blue, Pararosaniline (Feulgen), PBFI, Phloxin B (Magdala Red), Phorwite AR, Phorwite BKL, Phorwite Rev, Phorwite RPA, Phosphine 3R, PKH26 (Sigma), PKH67, PMIA, Pontochrome Blue Black, POPO-1, POPO-3, PO-PRO-1, PO-PRO-3, Primuline, Procion Yellow, Propidium lodid (PI), PyMPO, Pyrene, Pyronine, Pyronine B, Pyrozal Brilliant Flavin 7GF, QSY 7, Quinacrine Mustard, Resorufin, RH 414, Rhod-2, Rhodamine, Rhodamine 110, Rhodamine 123, Rhodamine 5 GLD, Rhodamine 6G, Rhodamine B, Rhodamine B 200, Rhodamine B extra, Rhodamine BB, Rhodamine BG, Rhodamine Green, Rhodamine Phallicidine, Rhodamine Phalloidine, Rhodamine Red, Rhodamine WT, Rose Bengal, S65A, S65C, S65L, S65T, SBFI, Serotonin, Sevron Brilliant Red 2B, Sevron Brilliant Red 4G, Sevron Brilliant Red B, Sevron Orange, Sevron Yellow L, SITS, SITS (Primuline), SITS (Stilbene Isothiosulphonic Acid), SNAFL calcein, SNAFL-1, SNAFL-2, SNARF calcein, SNARF1, Sodium Green, SpectrumAqua, SpectrumGreen, SpectrumOrange, Spectrum Red, SPQ (6-methoxy-N-(3-sulfopropyl)quinolinium), Stilbene, Sulphorhodamine B can C, Sulphorhodamine Extra, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, Tetracycline, Tetramethylrhodamine (TAMRA), Texas Red, Texas Red-X conjugate, Thiadicarbocyanine (DiSC3), Thiazine Red R, Thiazole Orange, Thioflavin 5, Thioflavin S, Thioflavin TCN, Thiolyte, Thiozole Orange, Tinopol CBS (Calcofluor White), TMR, TO-PRO-1, TO-PRO-3, TO-PRO-5, TOTO-1, TOTO-3, TRITC (tetramethylrodamine isothiocyanate), True Blue, TruRed, Ultralite, Uranine B, Uvitex SFC, WW 781, X-Rhodamine, XRITC, Xylene Orange, Y66F, Y66H, Y66W, YO-PRO-1, YO-PRO-3, YOYO-1, YOYO-3, SYBR Green, Thiazole orange (interchelating dyes), or combinations thereof.

In one aspect, the present disclosure is generally directed to systems and methods for amplifying the signal of targets (potentially tens, hundreds, thousands, or more) within a biological sample, e.g., for imaging using MERFISH or other techniques. For example, in some embodiments, these techniques provide a fast, simple, and/or efficient way to amplify the signal of hundreds or thousands of RNA targets simultaneously, e.g., in the native environment of biological samples. Such amplification can be well-controlled by using a saturable system, in certain embodiments, as discussed herein. Because of this, the variation in brightness from spot to spot can be minimized during the amplification, which can be useful in decoding using MERFISH or other techniques. In some embodiments, the sizes of the amplified spots also do not increase. This may improve the ability to identify targets, e.g., located relatively to close to each other. For example, the signal from one target may overlap with that from another target if the spot sizes increase too much. In addition, as discussed below, the amplifier nucleic acids in some embodiments do not contain hairpin structures, e.g., that may be involved in the amplification process, which may facilitate the creation of a saturable system, and/or apply the design of multiple amplifier systems to a large number of targets. In addition, also as discussed below, the amplifier nucleic acids may be constructed using only three nucleotides. Three-letter nucleotides may have significantly less secondary structure than four-letter nucleotides, and faster binding rates. In addition, in some cases, the possibility that any given amplifier sequence will work reliably is increased, e.g., by reducing the possibility of unintentional secondary structure.

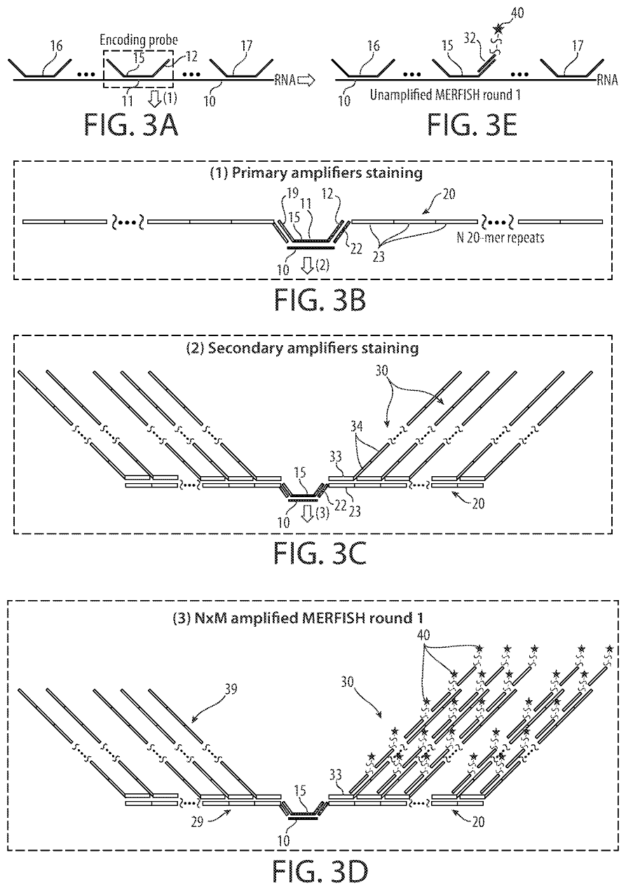

A non-limiting example of such a system is now illustrated in FIG. 3. In FIG. 3A, a target 10 (RNA, in this example) is illustrated. There may be hundreds or thousands of targets distributed within a biological sample (e.g., a cell or a tissue), and the binding of nucleic acid probes to the targets may be used to determine their distribution, e.g., by using fluorescent probes and imaging the sample. Note, however, that only a single target is illustrated here for clarity.

In some embodiments, a plurality of nucleic acid probes are used that have different sequences, and the distribution of each of the nucleic acid probes is sequentially analyzed and used to create “codewords” for each location, based on the binding patterns of each of the nucleic acid probes. By selecting nucleic acid probes that defines a suitable code space, apparent errors in the observed binding patterns can be identified, and/or discarded and/or corrected to identify the correct codeword, and thus the correct target of the nucleic acid probes within the sample. This error-robustness and error-correction system was first introduced for multiplexed error-robust fluorescence in situ hybridization (MERFISH), and has also been subsequently used in various related techniques. See, e.g., Int. Pat. Apl. Pub. Nos. WO 2016/018960 and WO 2016/018963, each incorporated herein by reference in its entirety.

An example of an encoding nucleic acid probe is shown in FIG. 3A, where an encoding nucleic acid probe 15 (shown in a dotted box) has bound to a target 10, e.g., a target RNA. Other nucleic acid probes 16, 17 may also bind to the target RNA, and/or to other targets within the sample. Probe 15 may comprise a target sequence 11 that is able to bind to the target RNA (e.g., via specific binding), and a read sequence 12 (or “readout” sequence), i.e., a sequence that can be “read” to determine whether or not binding has occurred. One, two, three, or more read sequences may be present on a probe. For instance, in this example, two such read sequences are present in probe 15 (identified as read sequence 12 and read sequence 19). The read sequences may each independently be the same or different. In addition, probes such as 16 and 17 may have the same or different numbers of read sequences, and/or the same or different structures, as nucleic acid probe 15.

If no amplification is applied, then nucleic acid probe 15 may be exposed to a suitable secondary nucleic acid probe 32 containing a signaling entity 40, as is shown in FIG. 3E. In this example, the signaling entity is linked to the secondary nucleic acid probe via a disulfide linkage, although other techniques may be used in other embodiments. However, in this case, only one signaling entity can be linked to the target. It thus can be relatively difficult to detect the single signaling entity, and use it to determine binding of nucleic acid probe 15 to target 10, due to the low signal intensity produced after such a binding event.

Accordingly, in FIG. 3B, a primary amplifier nucleic acid 20 can be used, in accordance with certain embodiments. The primary amplifier nucleic acid may contain a first primary recognition sequence 22 able to bind (e.g., specifically) to a read sequence of nucleic acid probe 15, and one or more primary read sequences 23 able to bind (e.g., specifically) to one or more secondary amplifier nucleic acids, as discussed below. In this example, “N” such read sequences are shown schematically in the primary amplifier nucleic acid (N may be, for instance, 5, 7, 9, or other numbers as discussed herein). The primary read sequences may each have the same or different sequences, and may have the same or different lengths. In this example, each read sequence is 20 nucleotides long, although this is by way of example only. In addition, as previously noted, although two such primary amplifier nucleic acids are shown here, this is by way of example only, and other numbers of primary amplifier nucleic acids may be bound to the nucleic acid probe in other embodiments.

Next, as shown in FIG. 3C, secondary amplifier nucleic acids 30 may be bound to the primary amplifier nucleic acid. A secondary amplifier nucleic acid may contain a first recognition sequence 33 able to bind (e.g., specifically) to read sequence 23 of primary amplifier nucleic acid 20, and one or more secondary read sequences 34 able to bind to a signaling entity, as discussed below.

As with the primary amplifier nucleic acids, any number of secondary read sequences may be present in the secondary amplifier nucleic acids, as is shown in this figure. The secondary read sequences may each have the same or different sequences, and may have the same or different lengths relative to each other. The secondary read sequences also may be the same or different than the read sequences of the primary amplifier nucleic acids. In this example, each secondary amplifier nucleic acid may have “M” read sequences. M may be, e.g., 5, 7, 9, or other numbers as discussed herein, and M may be the same or different than N.

In FIG. 3D, a plurality of signaling entities 40 has been bound to the read sequences of the secondary amplifier nucleic acids. In this example, the signaling entities are each bound via a disulfide linkage, although other techniques may be used in other embodiments, as discussed herein.

Additionally, in this case, there is expected to be a maximum, or a saturation limit, of signaling entities that have bound to each read sequence of the target sequence. In this particular example, there are N×M such positions available for each of the read sequences of the nucleic acid probe (2 such read sequences here), assuming both have substantially the same structure (although they do not necessarily have to have the same structure, i.e., the same number of N×M positions available, e.g., if they have amplifier nucleic acids with different structures). Thus, the number of signaling entities that can be associated with a given target is a finite, predictable number, and cannot grow indefinitely or without bound.

In this example, two read sequences 12 and 19 were discussed, each of which may have primary and secondary amplifier nucleic acids and associated signaling entities. These may or may not have the same or different structures, e.g., signaling entities and/or amplifier nucleic acids associated with read sequence 12 may not associate with read sequence 19, and vice versa. (However, as mentioned above, 2 read sequences are provided here by way of example only, and in other embodiments, there may be 1, 2, 3, 4, etc. distinct read sequences, which can be amplified in parallel, e.g., using similar approaches, including with distinct amplifier nucleic acids, etc.) The read sequences may be independently determined, e.g., sequentially or simultaneously, by determining signaling entities associated with each of the read sequences, which may be the same or different. For instance, as shown in FIG. 3D, signaling entities 40 are able to associate with primary amplifier nucleic acid 20 and secondary amplifier nucleic acid 30, ultimately to read sequence 12, but are not able to associate with read sequence 19 or its associated primary and secondary amplifier nucleic acids 29 and 39, respectively.

In addition, in some embodiments, amplification can involve the binding of only one round of amplifier nucleic acids (producing a N-fold amplification), two rounds (producing N×M-fold amplification), three rounds (producing a N×M×O amplification where the third round of molecules contain O read sequences), or more in some cases. In some cases, any number of rounds of amplification can be applied.

Additionally, the sample may contain nucleic acid probes that have many different readout sequences, e.g., that can be recognized using different amplifier nucleic acids or signaling entities. For example, 8, 10, 12, 14, 16, 24, 32, 48, 64, 128, or other numbers of readout sequences could be used, including more than 128 rounds. In some cases, unique amplifier nucleic acids can be used for the amplification of the readout sequences, e.g., such as that the original readout sequence present can be amplified into, e.g., N×M copies, using suitable amplifier nucleic acids. In certain cases, the amplifier nucleic acids may be efficiently designed. For example, by utilizing only three of the four nucleotides in the sequences of the amplifier nucleic acids, the probability of unanticipated secondary structure within the amplifier nucleic acids can be reduced. Without wishing to be bound by any theory, it is believed that because the effect of such secondary structure of binding of the amplifier nucleic acids themselves, or subsequent amplifier nucleic acids can be difficult to predict, reducing or eliminating secondary structure can increase the probability that a given amplifier nucleic acid will properly assemble, which can facilitate the design and use of large numbers of orthogonal amplifier nucleic acids. In addition, by reducing secondary structure, which can also inhibit the rate of binding of these amplifier nucleic acids, such design considerations can decrease the time required to assemble such structures, or decrease the amount of amplifier nucleic acids required for each sample, etc.

The controlled amplification provided by this approach is in contrast to techniques such as hairpin unfolding or rolling circle amplification, where amplification of a signal can effectively grow in an uncontrolled manner or indefinitely, i.e., when sufficient reagents are present. Such uncontrolled amplification can be difficult to accurately determine, as the amount of signal present may not be well-correlated to the number of targets, or the location of the target (for example, with larger amounts of signal created by uncontrolled amplification, the “spot size” appearing in a microscopic image may grow larger, and not necessarily centered around the target, thus impeding the resolution of the image, or interfering with signals from other, nearby targets). In contrast, the use of saturable amplification techniques, as discussed herein, may create a maximum number of signaling entities that can associate with a target, which may limit spot sizes, create uniformity in the brightness or intensities of the spots, improve detection, or the like.

As mentioned, in certain embodiments, such techniques may be combined with error correction, e.g., as is used in MERFISH or other similar techniques. For example, codewords may be based on the binding (or non-binding) of the plurality of nucleic acid probes, and in some cases, the codewords may define an error-correcting code to help reduce or prevent misidentification of the nucleic acid probes. In some cases, a relatively large number of different targets may be identified using a relatively small number of labels, e.g., by using various combinatorial approaches. Image acquisition techniques such as STORM can also be used to image such samples and facilitate determination of the nucleic acid probes. See, e.g., U.S. Pat. Nos. 9,712,805 or 10,073,035, or Int. Pat. Apl. Pub. Nos. WO 2008/091296 or WO 2009/085218, each incorporated herein by reference in its entirety, for additional details regarding techniques such as MERFISH.

Another aspect of the disclosure is directed to a computer-implemented method. For instance, a computer and/or an automated system may be provided that is able to automatically and/or repetitively perform any of the methods described herein. As used herein, “automated” devices refer to devices that are able to operate without human direction, i.e., an automated device can perform a function during a period of time after any human has finished taking any action to promote the function, e.g. by entering instructions into a computer to start the process. Typically, automated equipment can perform repetitive functions after this point in time. The processing steps may also be recorded onto a machine-readable medium in some cases.

For example, in some cases, a computer may be used to control imaging of the sample, e.g., using fluorescence microscopy, STORM or other super-resolution techniques such as those described herein. In some cases, the computer may also control operations such as drift correction, physical registration, hybridization and cluster alignment in image analysis, cluster decoding (e.g., fluorescent cluster decoding), error detection or correction (e.g., as discussed herein), noise reduction, identification of foreground features from background features (such as noise or debris in images), or the like. As an example, the computer may be used to control activation and/or excitation of signaling entities within the sample, and/or the acquisition of images of the signaling entities. In one set of embodiments, a sample may be excited using light having various wavelengths and/or intensities, and the sequence of the wavelengths of light used to excite the sample may be correlated, using a computer, to the images acquired of the sample containing the signaling entities. For instance, the computer may apply light having various wavelengths and/or intensities to a sample to yield different average numbers of signaling entities in each region of interest (e.g., one activated entity per location, two activated entities per location, etc.). In some cases, this information may be used to construct an image and/or determine the locations of the signaling entities, in some cases at high resolutions, as noted above.

In some aspects, the sample is positioned on a microscope. In some cases, the microscope may contain one or more channels, such as microfluidic channels, to direct or control fluid to or from the sample. For instance, in one embodiment, nucleic acid probes such as those discussed herein may be introduced and/or removed from the sample by flowing fluid through one or more channels to or from the sample. In some cases, there may also be one or more chambers or reservoirs for holding fluid, e.g., in fluidic communication with the channel, and/or with the sample. Those of ordinary skill in the art will be familiar with channels, including microfluidic channels, for moving fluid to or from a sample.

The following documents are incorporated herein by reference: Int. Pat. Apl. Pub. Nos. WO 2018/218150, entitled “Systems and Methods for High-Throughput Image-Based Screening”; WO 2016/018960, entitled “Systems and Methods for Determining Nucleic Acids”; WO 2016/018963, entitled “Probe Library Construction”; WO 2018/089445, entitled “Matrix Imprinting and Clearing”; WO 2018/089438, entitled “Multiplexed Imaging Using MERFISH and Expansion Microscopy”; and U.S. Pat. Apl. Ser. Nos. 62/836,578, entitled “Imaging-Based Pooled CRISPR Screening” and 62/779,333, entitled “Amplification Methods and Systems for MERFISH and Other Applications.” The following documents are also incorporated herein by reference in their entireties: U.S. Pat. Nos. 2017/0220733 and 2017/0212986. Furthermore, U.S. Pat. Apl. Ser. No. 62/938,194, filed Nov. 20, 2019, entitled “Systems and Methods for Multi-Focal Imaging for Molecular Profiling,” by Moffitt, et al., is incorporated herein by reference in its entirety.

The following examples are intended to illustrate certain embodiments of the present disclosure, but do not exemplify the full scope of the disclosure.

### EXAMPLE 1

To further illustrate the potential decrease in MERFISH imaging time provided by simultaneous multi-plane imaging in combination with a MERFISH measurement, this example provides the following two scenarios.

In the first scenario, an 8-image z-stack comprised of focal planes separated by 1.5 microns is used to characterize the distribution of numerous mRNA molecules at multiple fields-of-view (FOV) in a tissue slice. Assuming an exposure time per image of 0.5 s and a refocusing time of 100 ms, 4.7 s will be required to collect each z-stack. If such measurements were performed at 1000 FOV and 16 rounds of imaging were performed to measure 16-bit barcodes, the total time required to image this sample across all rounds would be ˜21 hours.

In the second scenario, one performs the same MERFISH measurement and images the same 8-image z-stacks in the same number of FOVs; however, one uses a camera bank comprised of 8 cameras with each camera in the bank imaging a focal plane 1.5-microns separated from the previous camera so that the imaged z-stack is identical to that imaged in the first scenario.

In addition, some embodiments use branched DNA amplification (bDNA) to amplify the fluorescence signal in each round by ˜8-fold, such that the total fluorescence on each camera in the bank is identical to the signal on the camera in the first scenario. In this scenario, each camera in the bank is exposed, simultaneously, for a total exposure time of 0.5 s. The objective is not refocused in this scenario. Thus, the time required to image the full z-stack at a single FOV is 0.5 s, and the time required to perform all imaging across 16 rounds in 1000 FOV would be 2.2 hours in this particular example.

### EXAMPLE 2

The example illustrates a third scenario in which four camera banks are used to image four different colors in the same tissue volume as described in Example 1. Like the second scenario described above, collection of a full z-stack would require only 0.5 s per FOV; however, by using four different color channels, four readout probes could be measured in each round of staining and imaging, and instead of requiring 16 rounds of imaging to measure 16 bits, only four rounds would be required. Thus, measurement of 16-bit barcodes across 1000 FOV would only require ˜33 minutes in this particular example.

### EXAMPLE 3

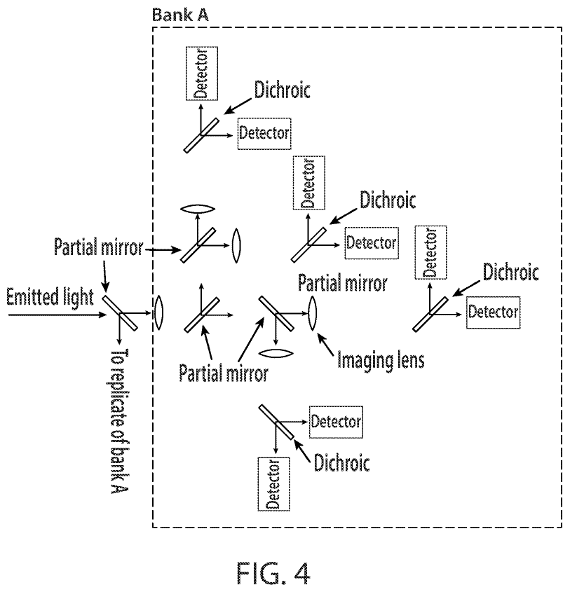

The example illustrates a scenario where a bank of 16 cameras are arranged to image 8 optical planes in two color channels simultaneously (FIG. 4). Emitted light collected from a microscope or some other imaging device is split by a non-dichroic beam splitter equally between two identical paths. In the first path, the light is focused with a first imaging lens, split across a series of two additional non-dichroic beam splitters (partial mirrors) to create four optical paths each containing, ideally, a roughly equal fraction of the emitted light collected from the microscope. In each of these paths, the light then passes through a second imaging lens to form an image on each of 8 detectors. However, before the light reaches a detector, it is split via color using a dichroic beamsplitter, creating an image in each of the color channels on two separate cameras. The position of each such pair of cameras separated by a dichroic beamsplitter is set relative to the final imaging lens in their shared path such that they are imaging the same axial plane in the sample imaged with the microscope to which this system is attached. However, the distance from the final imaging lens associated with each of pair of cameras to that pair of cameras is unique for each camera pair, so that each pair is imaging a different axial plane in the sample relative to the other camera pairs. The second optical path created by the first non-dichroic beam splitter contains an equivalent path.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

All numbers herein are approximate and are intended to account for both normal measurement errors as well as rounding to the nearest significant figure.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
exposing a sample to a plurality of nucleic acid probes;
for each of the nucleic acid probes, capturing images of the sample using at least 2 detectors focused on different focal planes within the sample;
determining binding of the nucleic acid probes within the sample using the images; and
determining an abundance and/or a spatial distribution of nucleic acids within the sample corresponding to the binding of the plurality of nucleic acid probes.

2. The method of claim 1, comprising capturing images of the sample using at least 4 detectors focused on different focal planes within the sample.

3. The method of any one of claim 1 or 2, comprising capturing images of the sample using at least 8 detectors focused on different focal planes within the sample.

4. The method of any one of claims 1-3, comprising capturing images of the sample using at least 16 detectors focused on different focal planes within the sample.

5. The method of any one of claims 1-4, comprising capturing images of the sample using at least 32 detectors focused on different focal planes within the sample.

6. The method of any one of claims 1-5, wherein each of the different focal planes is focused at no more than 1000 nm from a neighboring focal plane.

7. The method of any one of claims 1-6, comprising passing light from the sample through a plurality of beamsplitters to the at least 4 detectors.

8. The method of any one of claims 1-7, wherein at least some of the detectors are cameras.

9. The method of any one of claims 1-8, wherein at least some of the detectors are point detectors.

10. The method of any one of claims 1-9, wherein at least some of the detectors are photodetectors.

11. The method of any one of claims 1-10, wherein at least some of the detectors are avalanche photodiodes.

12. The method of any one of claims 1-11, wherein at least some of the detectors are one dimensional arrays.

13. The method of any one of claims 1-12, wherein at least some of the detectors are line detectors.

14. The method of any one of claims 1-13, comprising determining z positions of at least some of the nucleic acid probes in the sample at a resolution better than 300 nm.

15. The method of any one of claims 1-14, comprising exposing the nucleic acid probes to primary amplifier nucleic acids able to bind to the nucleic acid probes, wherein a maximum number of primary amplifier nucleic acids is able to bind to a nucleic acid probe; and
exposing the primary amplifier nucleic acids to secondary amplifier nucleic acids able to bind to the primary amplifier nucleic acids, wherein a maximum number of secondary amplifier nucleic acids is able to bind to the primary amplifier nucleic acids.

16. The method of claim 15, comprising exposing the primary amplifier nucleic acids to secondary amplifier nucleic acids able to bind to the primary amplifier nucleic acids, wherein the binding of primary amplifier nucleic acids and secondary amplifier nucleic acids to a target is saturable.

17. The method of any one of claim 15 or 16, comprising exposing the primary amplifier nucleic acids to secondary amplifier nucleic acids able to bind to the primary amplifier nucleic acids, wherein the secondary amplifier nucleic acids bind to the primary amplifier nucleic acids within a fixed distance.

18. The method of any one of claims 15-17, wherein no more than 20 primary amplifier nucleic acids is able to bind to the nucleic acid probe.

19. The method of any one of claims 15-18, wherein the primary amplifier nucleic acids have an average length of less than 300 nucleotides.

20. The method of any one of claims 15-19, further comprising exposing the primary amplifier nucleic acids to secondary amplifier nucleic acids able to bind to the primary amplifier nucleic acids, wherein a maximum number of secondary amplifier nucleic acids is able to bind to the primary amplifier nucleic acids.

21. The method of claim 20, wherein the secondary amplifier nucleic acids comprise a fluorescent signaling entity.

22. The method of any one of claims 1-21, further comprising:
creating codewords binding of the nucleic acid probes within the sample; and
for at least some of the codewords, matching the codeword to a valid codeword optionally wherein, if no match is found, applying error correction to the codeword to form a valid codeword.

23. The method of any one of claims 1-22, comprising exposing the sample to at least 5 different nucleic acid probes.

24. The method of any one of claims 1-23, comprising sequentially exposing the sample to a plurality of nucleic acid probes.

25. The method of any one of claims 1-24, wherein the plurality of nucleic acid probes comprises a combinatorial combination of nucleic acid probes with different sequences.

26. The method of any one of claims 1-25, wherein the plurality of nucleic acid probes have an average length of between 10 and 300 nucleotides.

27. The method of any one of claims 1-26, wherein at least some of the plurality of nucleic acid probes comprises a target sequence and one or more read sequences.

28. The method of claim 27, wherein the target sequence of the plurality of nucleic acid probes has an average length of between 10 and 200 nucleotides.

29. The method of any one of claim 27 or 28, wherein the target sequence binds to a target via specific binding.

30. The method of any one of claims 27-29, wherein the plurality of read sequences are distributed on the plurality of nucleic acid probes so as to define an error-correcting code.

31. The method of any one of claims 1-30, wherein the plurality of nucleic acid probes defines a code space with a Hamming distance of at least 2.

32. The method of any one of claims 1-31, comprising determining binding of the nucleic acid probes using fluorescence imaging.

33. The method of any one of claims 1-32, comprising determining binding of the nucleic acid probes using multi-color fluorescence imaging.

34. The method of any one of claims 1-33, comprising determining binding of the nucleic acid probes using a super-resolution fluorescence imaging technique.

35. The method of claim 34, comprising determining binding of the nucleic acid probes using stochastic optical reconstruction microscopy (STORM).

36. The method of any one of claims 1-35, wherein the sample comprises a cell.

37. The method of claim 36, wherein the cell is a human cell.

38. The method of any one of claim 36 or 37, wherein the cell is fixed.

39. A method, comprising:
exposing a sample to a plurality of nucleic acid probes;
for each of the nucleic acid probes, exposing the nucleic acid probes to amplifier nucleic acids able to bind thereto, wherein a maximum finite number of amplifier nucleic acids is able to directly or indirectly bind to a nucleic acid probe; and
for each of the nucleic acid probes, capturing images of the sample using at least 4 cameras focused on different focal planes within the sample.
